SEQUENCE LISTING (15 sequences)

SEQ ID NO: 1 - protein, 247 aa, Staphylococcus aureus
MKNKLLFKIFLSLSLALSVYSINDKIIEVSNTSLAADVKNFTDLDEATKWGNKLIKQAKYSSDDKIALYEYTKDSSKINGPLRLAGGDINKLDSTTQDKVRRLDSSISKSTTPESVYVYRLLNLDYLTSIVGFTNEDLYKLQQTNNGQYDENLVRKLNNVMNSRIYREDGYSSTQLVSGAAVGGRPIELRLELPKGTKAAYLNSKDLTAYYGQQEVLLPRGTEYAVGSVELSNDKKKIIITAIVFKK

SEQ ID NO: 2 - protein, 340 aa, Saccharomyces cerevisiae
MNIKDRTSEFQQSVLSYKKRNKNFREQQRERLQEKESENFANNTTGNGKSVSEFQKKASGIAHEISSTAQLLSKLAVLAKRKPMFNDNPVEIAELSFLIKRKIYAIEQSLVQLSQLKKTDVNGNTSNQSSKQPSAVQHSKNVVNLLNTQMKNISGSFKDVLEERQRLEMANKDRWQKLTTDTGHAPADDQTQSNHAADLTTYNNSNPFMTSLLDESSEKNNNSSNQGELSFPQNDSQLMLMEEGQLSNNVYLQERNRAVETIESTIQEVGNLFQQLASMVQEQGEVIQRIDANVDDIDLNISGAQRELLKYFDRIKSNRWLAAKVFFIIFVFFVIWVLVN

SEQ ID NO: 3 - DNA, 28 bp, Artificial
  Synthetic primer
gatactcgaggtcgggagttcgagacca

SEQ ID NO: 4 - DNA, 28 bp, Artificial
  Synthetic primer
gtttaagcttactcgcgttgttcccgcg

SEQ ID NO: 5 - DNA, 20 bp, Artificial
  Synthetic primer
gctgagcgtataaagacacc

SEQ ID NO: 6 - DNA, 20 bp, Artificial
  Synthetic primer
tctacatcctcagtgggaac

SEQ ID NO: 7 - DNA, 18 bp, Artificial
  Synthetic primer
tcatgcaacacccaacac

SEQ ID NO: 8 - DNA, 18 bp, Artificial
  Synthetic primer
cacaaccggtgtttcctc

SEQ ID NO: 9 - DNA, 20 bp, Artificial
  Synthetic primer
catcagggagctgcagtaca

SEQ ID NO: 10 - DNA, 20 bp, Artificial
  Synthetic primer
cccagcttcaccttcttcag

SEQ ID NO: 11 - DNA, 21 bp, Artificial
  Synthetic primer
catgggagggcttgatgaagg

SEQ ID NO: 12 - DNA, 21 bp, Artificial
  Synthetic primer
caaggtcagcatggactaggc

SEQ ID NO: 13 - DNA, 21 bp, Artificial
  Synthetic primer
aattctccgaacgtgtcacgt

SEQ ID NO: 14 - DNA, 21 bp, Artificial
  Synthetic primer
aatgacatgccgatctacatg

SEQ ID NO: 15 - DNA, 21 bp, Artificial
  Synthetic primer
gcacaagtcatcccgttaatt

(19) United States
(12) Patent Application Publication
(10) Pub. No.: US 2016/0375050 A1
(43) Pub. Date: Dec. 29, 2016

(54) METHODS AND COMPOSITIONS INVOLVING CHITOSAN NANOPARTICLES

(71) Applicant: The Board of Regents of the University of Texas System, Austin, TX (US)

(72) Inventors: Gabriel LOPEZ-BERESTEIN, Bellaire, TX (US); Eylem GÜVEN, Ankara (TR); Angela SANGUINO, Pittsburgh, PA (US); Anil K. SOOD, Pearland, TX (US); Emir Baki DENKBAS, Ankara (TR)

(73) Assignee: The Board of Regents of the University of Texas System, Austin, TX (US)

(21) Appl. No.: 15/264,551

(22) Filed: Sep. 13, 2016

Related U.S. Application Data
(62) Division of application No. 12/680,728, filed on Dec. 1, 2010.
(60) Provisional application No. 60/978,353, filed on Oct. 8, 2007.

(51) Int. Cl. A61K 31/722; A61K 47/02; A61K 31/713; A61K 31/575; C12N 15/113; A61K 45/06

(52) U.S. Cl. CPC A61K 31/722; C12N 15/1137; C12Y 201/01043; A61K 45/06; A61K 31/713; A61K 31/575; A61K 47/02; C12N 2310/14; C12N 2320/31; C12N 2320/32

(57) ABSTRACT

Disclosed are nanoparticles for the delivery of a therapeutic agent or a diagnostic agent to a subject that include a chitosan and a polyphosphate, wherein the weight ratio of the chitosan to the polyphosphate is about 1.0 or greater and the weight ratio of the polyphosphate to the therapeutic agent or diagnostic agent is about 15.0 or less. Also disclosed are nanoparticles that include a chitosan and an inhibitor of enhancer of Zeste homologue 2 (EZH2). Methods of delivering a therapeutic agent or a diagnostic agent to a subject for the treatment or prevention of a disease and methods of predicting prognosis of ovarian cancer in a subject that involve determining the expression and/or function of EZH2 in the subject are also disclosed.

The present application is a divisional of U.S. application Ser. No. 12/680,728, filed Dec. 1, 2010, which is a national phase application filed under 35 U.S.C. §371 of International Application No. PCT/US2008/079212, filed Oct. 8, 2008, which claims the benefit of priority to U.S. Provisional Patent Application No. 60/978,353, filed Oct. 8, 2007, the entire contents of each of which are hereby specifically incorporated by reference.

### BACKGROUND OF THE INVENTION

1. Field of the Invention

The present invention generally relates to the fields of molecular biology, pharmaceutics, and oncology. More particularly, the invention concerns nanoparticles comprising a chitosan, a polyphosphate, and a therapeutic agent or diagnostic agent, wherein the weight ratio of the chitosan to the polyphosphate is about 1.0 or greater and the weight ratio of the polyphosphate to the therapeutic agent or diagnostic agent is about 15.0 or less, and nanoparticles that include a chitosan and an inhibitor of enhancer of Zeste homologue 2 (EZH2). The invention also concerns methods of delivery of a therapeutic agent or diagnostic agent into a subject employing nanoparticles of the present invention. The invention further concerns methods of predicting prognosis of ovarian cancer in a subject that involve determining the expression and/or function of EZH2 in the subject.

2. Description of Related Art

Cancer is a major cause of morbidity and mortality in the U.S. Regarding ovarian cancer, mortality rates remain high despite substantial improvements in surgical and chemotherapeutic treatment approaches; thus, novel treatment strategies are urgently needed. Targeting the tumor vasculature is a particularly attractive strategy because of the presumed genetic stability of endothelial cells (Folkman et al., 1990). The recent success of anti-angiogenic therapy with bevacizumab in patients with solid tumors has confirmed the clinical viability of this approach (Jain et al., 2006; Burger et al., 2007; Spannuth et al., 2008). However, despite initial responses, most patients eventually experience disease progression; therefore, new anti-angiogenesis targets are needed.

The enhancer of Zeste homologue 2 (EZH2) is a member of the polycomb-group (PcG) proteins. PcG proteins are negative regulators of gene expression and are involved in the stable transmission of the repressive state of their target gene throughout the cell cycle (Simon, 1995; Cavalli and Paro, 1998; Kingston et al., 1996). EZH2, a critical component of the polycomb repressive complex 2 (PRC2), has intrinsic histone methyl transferase (HMTase) activity and has been implicated in the progression and metastasis of several cancers (Raman et al., 2005; Cha et al., 2005) but its precise role remains unknown.

While a number of attractive targets in tumor and endothelial cells have been identified, many of these are difficult to target with conventional approaches such as small molecule inhibitors or monoclonal antibodies. Small interfering RNA (siRNA)-based approaches may allow development of a broader armamentarium of targeted drugs. However, to achieve therapeutic success, several hurdles must be overcome including rapid clearance, nuclease mediated degradation, systemic in vivo delivery and intracellular localization. It has been recently demonstrated that a neutral nanoliposomal carrier allows efficient systemic delivery of siRNA into orthotopic tumors (Landen et al., 2005; Thaker et al., 2006).

Chitosan (CH) is a naturally occurring polysaccharide that is attractive for biological applications due to properties such as low immunogenicity and low toxicity (Kumar, 2000). A chitosan is a cationic polysaccharide derived from chitin, which is a copolymer of glucosamine and N-acetyl glucosamine units (Mi et al., 1999; Gupta and Ravi Kumar, 2001; Kumar, 2000). Chitosans have been evaluated as carriers for drugs in nanoparticles in view of their biocompatilibity and biodegradability (Bayomi et al., 1998; Genta et al., 1998; Ko et al., 2003; Katas and Alpar, 2006).

Nanoparticle delivery of therapeutic agents is an area of active investigation. Traditional drug delivery methods include oral and intravenous routes of administration. These methods are still the most widely used today, yet each has its disadvantages. Oral delivery via tablets or capsules is often ineffective due to exposure of the pharmaceutical agent to the metabolic processes of the body. Therefore, a larger than necessary dose is often required and the maximum effectiveness of the drug is limited. Intravenous administration is often problematic. Specificity for injectable agents is often low, requiring injection of large amounts of the agent, creating a high concentration of the drug in the blood stream that can lead to toxic side effects. There have been limited reports concerning nanoparticles that include chitosan and TPP for delivery of siRNA (Katas and Alpar, 2006; Liu et al., 2007).

Thus, there is the need for more effective methods of delivering therapeutic agents to target tumor cells in a subject.

### SUMMARY OF THE INVENTION

The present invention provides for drug delivery particles that include a chitosan and a polyphosphate which can be applied in effective delivery of therapeutic agents and diagnostic agents into tissues of a subject. For example, the inventors have found that nanoparticles that are composed of chitosan and tripolyphosphate anion can be applied in the successful delivery of siRNA into tissues of a subject.

Some aspects of the present invention generally pertain to nanoparticles for delivery of a therapeutic agent or diagnostic agent that include: (a) a chitosan, (b) a polyphosphate anion of formula (I):

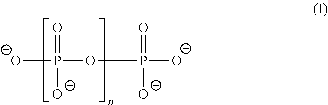

wherein n is an integer ranging from 2-10; and; (c) a therapeutic agent or a diagnostic agent, wherein the weight ratio of the chitosan to the polyphosphate is about 1.0 or greater and the weight ratio of the polyphosphate to the therapeutic agent or diagnostic agent is about 15.0 or less. Nanoparticles are generally defined as particles between 10 nanometers (nm) and 1000 nm in size, and can be either spherical or vesicular.

The term “chitosan,” as used herein, will be understood by those skilled in the art to include all derivatives of chitin, or poly-N-acetyl-D-glucosamine (including all polyglucosamine and oligomers of glucosamine materials of different molecular weights), in which the greater proportion of the N-acetyl groups have been removed through hydrolysis (that is greater than about 50% deacetylation: for example, about 51%, 55%, 60%, 65%, 70%, 75%, 80%, 85%, 90%, 95%, 99% or more deacetylated, or any range derivable therein). Typically, the chitosan is a cation. In some embodiments, the chitosan has a deacetylation degree of greater than about 50%. In more particular embodiments, the chitosan has a deacetylation degree of greater than about 60%. In more particular embodiments, the chitosan has a deacetylation degree of greater than about 70%. In even more particular embodiments, the chitosan has a deacetylation degree of greater than about 80%. In particular embodiments, the chitosan has a deacetylation degree of about 75% to about 85%.

The chitosan can be of any viscosity, but in particular embodiments it has a viscosity of about 20 cP to about 200 cP. The nanoparticle may include a single specific chitosan species, or more than one chitosan species.

Regarding the polyphosphate, in particular embodiments n is an integer ranging from 2 to 4. In particular embodiments, n is 3, and the polyphosphate anion is tripolyphosphate anion.

In particular embodiments, the weight ratio of the chitosan to the polyphosphate is about 2 to about 10. In more particular embodiments, the weight ratio of the chitosan to the polyphosphate is about 2 to about 6. In even more particular embodiments, the weight ratio of the chitosan to the polyphosphate is about 2 to about 4. In particular embodiments, the weight ratio of the chitosan to the polyphosphate is about 2.5 to about 3.5.

In specific embodiments, the weight ratio of the polyphosphate to the diagnostic agent or therapeutic agent is about 1 to about 14. In more specific embodiments, the weight ratio of the polyphosphate to the diagnostic agent or therapeutic agent is about 4 to about 12. In further specific embodiments, the weight ratio of the polyphosphate to the diagnostic agent or therapeutic agent is about 8 to about 12. In even more specific embodiments, the weight ratio of the polyphosphate to the diagnostic agent or therapeutic agent is about 8 to about 12, n is 3, and the weight ratio of the chitosan to the polyphosphate is about 2 to about 4.

The diagnostic agent or therapeutic agent can be any diagnostic agent or therapeutic agent known to those of ordinary skill in the art. A “diagnostic agent” is defined herein to refer to any agent that can be applied in the diagnosis of a disease or health-related condition. “Diagnosis” as used herein refers to an assessment of the presence of a disease or of the progression of a disease. A “therapeutic agent” is defined herein to refer to any agent that can be applied in the treatment of a disease or health-related condition. The diagnostic or therapeutic agent may be any molecule, such as a small molecule, a peptide, a polypeptide, a protein, an antibody, an antibody fragment, a DNA, or a RNA. Examples of such agents are set forth in the specification below.

In particular embodiments, the diagnostic or therapeutic agent is an inhibitor of enhancer of Zeste homologue 2 (EZH2). An “inhibitor” as used herein may refer to an agent that reduces the function of EZH2 or inhibits the expression of a gene that encodes EZH2. The inhibitor may function directly or indirectly to inhibit EZH2. The inhibitor may be a small molecule, a peptide, a polypeptide, a protein, an antibody, an antibody fragment, a DNA, or a RNA. In particular embodiments, the inhibitor is a nucleic acid that inhibits the expression of a gene that encodes EZH2, such as a siRNA.

In some embodiments, the nanoparticle includes one or more therapeutic agents. In other embodiments, the nanoparticle includes one or more diagnostic agents. In further embodiments, the nanoparticle includes one or more diagnostic agents and one or more therapeutic agents. In specific embodiments, the therapeutic agent is a RNA, such as a siRNA. Interference RNA and siRNA are discussed in greater detail in the specification below.

It is contemplated that the chitosan may or may not be ionically or covalently bonded to the polyphosphate anion. In those embodiments wherein the chitosan becomes bonded ionically or covalently to a polyphosphate anion, a “weight ratio” of chitosan to polyphosphate anion is contemplated to refer to the ratio of the weight of the chitosan component to the weight of the polyphosphate anion component of the nanoparticle. In some embodiments, for example, the nanoparticle includes a compound of formula (II):

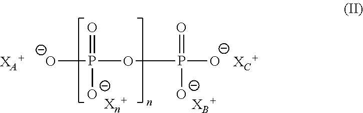

wherein n is an integer ranging from 2-10; X_{A}, X_{B}, X_{C} and Xₙ are each independently a cation selected from the group consisting of a chitosan H⁺, Na⁺, K⁺, Cs⁺, and NH₄⁺, and at least one of X_{A}, X_{B}, X_{C} and Xₙ is a chitosan. Thus, for example, in an embodiments wherein the only chitosan is X_{A}, the weight ratio of the chitosan to the polyphosphate would be the ratio of the weight of X_{A} to the weight of the compound of formula (II) excluding the weight of X_{A}.

Other aspects of the present invention include therapeutic nanoparticles that include an inhibitor of EZH2. In particular embodiments the inhibitor of EZH2 is a nucleic acid component that includes a nucleic acid that inhibits the expression of a gene that encodes EZH2. The nucleic acid may optionally encode a secondary therapeutic agent that can be applied in the treatment of a disease. For example, in some embodiments the nucleic acid component includes a secondary therapeutic agent that is an inhibitor of vascular endothelial growth factor (VEGF). Non-limiting examples of VEGF inhibitors include antibodies. For example, the antibody may be a monoclonal antibody, such as bevacizumab. In some embodiments, the nanoparticle further includes a polyphosphate. For example, the polyphosphate may be a polyphosphate of formula (I), (II), or any polyphosphate previously set forth. In specific embodiments, the polyphosphate is of formula (I) and n is 3. The therapeutic nanoparticles may optionally include additional components, such as cholesterol or a secondary therapeutic agent.

The present invention also generally pertains to methods of delivering a therapeutic agent or diagnostic agent to a subject, comprising administering to the subject a pharmaceutical composition comprising any nanoparticle as set forth herein.

The subject can be any subject, such as a mammal. For example, the subject may be a human, a mouse, a rat, a rabbit, a dog, a cat, a cow, a horse, a pig, a goat, a sheep, a primate, or an avian species. In particular embodiments, the subject is a human. For example, the human may be a subject with a disease. The disease may be any disease that afflicts a subject, such as an inflammatory disease, a hyperproliferative disease, an infectious disease, or a degenerative disease. In particular embodiments, the disease is a hyperproliferative disease such as cancer. For example, the cancer may be breast cancer, lung cancer, prostate cancer, ovarian cancer, brain cancer cell, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, intestinal cancer, lymphoma, or leukemia. In particular embodiments, the cancer is ovarian cancer.

The therapeutic agent or diagnostic agent may be any such agent known to those of ordinary skill in the art, such as any of those agents discussed above. For example, the therapeutic or diagnostic agent may be a small molecule, a peptide, a protein, a polypeptide, an antibody, an antibody fragment, a DNA or a RNA. In some embodiments, the therapeutic or diagnostic agent is a siRNA. In some embodiments, the therapeutic agent is an inhibitor of EZH2.

The present invention also concerns methods of preparing a nanoparticle, involving the steps of: (a) preparing a composition comprising a chitosan and a solvent; (b) adjusting the pH of the composition of (a) to a pH of greater than 3.0; and (c) adding a polyphosphate of formula (II) to the composition of (b):

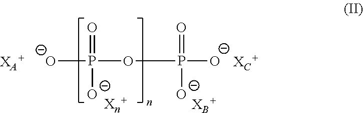

wherein n is an integer ranging from 2-10; and X_{A}, X_{B}, X_{C} and Xₙ are each independently a monovalent cation selected from the group consisting of H⁺, Na⁺, K⁺, Cs⁺, and NH₄⁺, wherein nanoparticles are formed.

The solvent can be any solvent, but in particular embodiments the solvent is an aqueous solvent. For example, the aqueous solvent may be water, acetic acid, or hydrochloric acid.

In certain specific embodiments, n is 3. In more specific embodiments, n is 3 and X_{A}, X_{B}, X_{C} and Xₙ are each Na⁺. In particular embodiments, the weight ratio of the chitosan to the polyphosphate is 1.0 or greater and the weight ratio of the polyphosphate to the therapeutic agent or diagnostic agent is 15.0 or less.

In further embodiments, the method involves the step of purifying the nanoparticles produced in (c). Purification can be by any method known to those of ordinary skill in the art. For example, purification may involve centrifuging the composition of (c), and removal of supernatant. Other methods for particle purification can be used, including high performance liquid chromatography (HPLC), gel permeation chromatography (GPC), and dialysis using a membrane filter.

In some embodiments, the method of producing a nanoparticle further involves adding a therapeutic agent or a diagnostic agent to the composition of (a), (b), or (c). For example, the therapeutic agent or diagnostic agent may be added to the composition of (b). The therapeutic agent or diagnostic agent can be any agent known to those of ordinary skill in the art. For example, the therapeutic agent or diagnostic agent may be any of those agents discussed above and elsewhere in this specification. In particular embodiments, the therapeutic or diagnostic agent is a siRNA. Detail regarding siRNA is discussed in the specification below. In some embodiments, a nucleic acid that inhibits the expression of a gene that encodes EZH2 is added to the composition of (a), (b), or (c).

The present invention also generally concerns methods of treating a subject with ovarian cancer that involve administering to a subject with ovarian cancer a pharmaceutically effective amount of a composition that includes a chitosan, and a nucleic acid component comprising a nucleic acid that inhibits the expression of a gene that encodes EZH2. Further embodiments concern methods of inhibiting angiogenesis in a subject that involve administering to a subject with angiogenesis a pharmaceutically effective amount of a composition that includes a chitosan, and a nucleic acid component comprising a nucleic acid that inhibits the expression of a gene that encodes EZH2.

In some embodiments, the nucleic acid component includes a siRNA or a nucleic acid encoding a siRNA, wherein the siRNA inhibits the expression of a gene that encodes EZH2 in the subject. In particular embodiments, the composition forms nanoparticles. The composition may optionally include one or more additional components. In some embodiments, the additional component is a lipid. Non-limiting examples of lipids include cholesterol, phosphatidylcholine, and phosphatidylethanolamine. In some embodiments, composition includes a polyphosphate or polyphosphate anion as discussed above. The subject can be any subject as discussed above, but in specific embodiments the subject is a human subject. Some embodiments further include identifying a subject in need of treatment of ovarian cancer. Identifying a subject in need can be by any method known to those of ordinary skill in the art. Examples include self-referral or diagnosing presence of ovarian cancer in the subject such as by physical examination, imaging techniques, and/or biopsy.

In some embodiments, the methods of the present invention further involve administering an additional anticancer therapy to the subject. For example, the additional anticancer therapy may be chemotherapy, radiation therapy, surgical therapy, immunotherapy, gene therapy, or a combination thereof. Non-limiting examples of chemotherapeutic agents include docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristine, vinblastin, and methotrexate. In particular embodiments, the chemotherapy is a VEGF inhibitor.

The composition may be administered to the patient by any method known to those of ordinary skill in the art. For example, the composition may be administered to the patient intravenously, intraperitoneally, intratracheally, intratumorally, intramuscularly, endoscopically, intralesionally, percutaneously, subcutaneously, regionally, or by direct injection or perfusion.

In some embodiments, the method is further defined as a method to inhibit growth of an ovarian cancer in a subject. In other embodiments, the method is further defined as a method to inhibit angiogenesis in an ovarian cancer in a subject.

Other aspects of the present invention concern methods to inhibit proliferation of an ovarian cancer cell involving contacting said cell with a composition that includes a chitosan and a nucleic acid component comprising a nucleic acid that inhibits the expression of a gene that encodes EZH2, wherein proliferation of the ovarian cancer cell is inhibited. The ovarian cancer cell may be in vivo (in a subject) or in vitro. In particular embodiments, the cancer cell is in a human subject. The composition may be a composition that includes nanoparticles of the present invention.

The invention further includes methods of predicting prognosis of a subject with an ovarian cancer that involve determining expression and/or function of EZH2 in ovarian cancer cells or ovarian cancer-associated endothelial cells in the subject, wherein increased EZH2 expression in said ovarian cancer cells or said endothelial cells is predictive of poor prognosis. In particular embodiments, the subject is a human subject. Determining expression and/or function of EZH2 may be by any method known to those of ordinary skill in the art. For example, determining expression and/or function of EZH2 may involve performing western blot analysis, immunohistochemistry, or protein array. In some embodiments, determining expression and/or function of EZH2 involves determining mRNA transcription as an indirect measure of EZH2 expression in said cell.

Some embodiments involve determining expression and/or function of EZH2 in normal cells (i.e., noncancerous cells) of said subject and comparing said expression and/or function of EZH2 in normal cells to said expression and/or function of EZH2 in ovarian cancer cells or ovarian cancer-associated endothelial cells. In such embodiments, increased expression and/or function of EZH2 in said ovarian cancer cells or ovarian cancer-associated endothelial cells compared to said expression and/or function of EZH2 in normal cells is predictive of poor prognosis. Poor prognosis may be reduced survival compared to a subject with greater expression and/or function of EZH2 in ovarian cancer cells or ovarian cancer-associated endothelial cells. The normal cells may be noncancerous cells from the subject, or noncancerous cells from a second subject without cancer. The normal cells may be ovarian cells, buccal mucosa cells, skin cells, or any other cell type that is noncancerous.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1A-E. Physical properties of nanoparticles. Physical characteristics of siRNA-incorporated chitosan particles. (A) the mean particles size, (B) zeta potential, (C) encapsulation efficiency of siRNA into chitosan particles, (D) siRNA incorporation into CH particles, and (E) morphology of siRNA-incorporated chitosan particles.

FIG. 2A-B. Stability of particles. (A) the electrophoretic migration of siRNA-chitosan particles in the presence of 50% FBS was visualized using a 4% agarose gel (100 V, 1 hr). Staining of the siRNA bound in chitosan particles indicate that the siRNA remains in the loading well without evidence for degradation, while the aqueous siRNA runs as a brightly stained band that diminishes with incubation time. Lane 1, naked siRNA; Lane 2-8, different time point incubated at 37° C. (B) The stability of the siRNA-chitosan particles against an exchange reaction by an anionic polymer, PLLA. The reaction mixture was evaluated by electrophoresis using a 4% agarose gel at 100 V for 1 hr.

FIG. 3A-C. Transfection efficiency of siRNA-chitosan particles. Cells, HeyA8 ovarian cancer cell line, were seeded at 1×10⁵ cells in a 6-well, and then siRNA alone, siRNA-chitosan particles, and RNAipec® as a positive control were added to each well without serum. After 4 hr incubation at 37° C., the cells were washed with serum-free media. The cells were fed again with RPMI 1640 media containing 10% (v/v) FBS, cultured for 24 hr after transfection, harvested with PBS buffer. (A) flow cytometry analysis to demonstrate the in vitro transfection of siRNA-chitosan particles, (B) Bar graph depicting the % of transfection efficiency of siRNA-chitosan particles into HeyA8 cells, (C) morphology of transfected siRNA-chitosan particles into HeyA8 cells.

FIG. 4. Micrographs showing the cellular distribution of the siRNA-chitosan tagged with Alexa-555 (particles in the cytoplasm) in the lumbar DRG and spinal dorsal horn 24 hr after intrathecal injection in one rat.

FIG. 5. Effect of intrathecal treatment with siRNA targeting the M2 subtype on the M2 mRNA level in the spinal cord and DRGs in rats. Intrathecal treatment with the control siRNA and M2 siRNA with two different sequences (5 μg every other day for 6 days, n=8-9 rats in each group) on the M2 mRNA level in the lumbar spinal cord and DRGs. The tissues were removed 3 days after the last treatment. The M2 mRNA level was quantified with the real-time RT-PCR and normalized to the endogenous reference gene (beta-actin).

FIG. 6. Effect of intrathecal treatment with the M2 siRNA on the M3 subtype mRNA level in the spinal cord and DRGs in rats. Intrathecal treatment with the control siRNA and M2 siRNA with two different sequences (5 μg every other day for 6 days, n=8-9 rats in each group) on the M3 mRNA level in the lumbar spinal cord and DRGs. Note that treatment with the M2 siRNA had no evident effect on the M3 mRNA level in both the spinal cord and DRGs.

FIG. 7. Effect of intrathecal M2 siRNA with two different sequences (5 μg every other day, n=8-9 rats in each group) on the M2 subtype protein level in the spinal cord, measured with [³H]QNB binding and immunoprecipitation 6 days after intrathecal treatment. Control, mismatch siRNA control.

FIG. 8. Effect of intrathecal treatment with the M2 siRNA (5 μg every other day for 6 days) on the analgesic effect of muscarine. The antinociceptive effect of intrathecal injection of 10 μg of muscarine in rats (n=8-9 in each group) 6 days after intrathecal treatment with M2 siRNA or mismatch control. Nociception was measured using a radiant heat stimulus.

FIG. 9A-H. EZH2 expression in human ovarian carcinoma. (A) Representative images of human tumors with low and high immunohistochemical staining for EZH2. (B) Kaplan-Meier curves of disease-specific mortality for patients whose ovarian tumors expressed high and low levels of EZH2 (EZH2-T). The log-rank test (two-sided) was used to compare differences between the two groups. Increased EZH2-T was significantly associated with decreased overall survival (p<0.001). (C) Representative images of human ovarian tumor vasculature (arrowheads point to endothelial cells) with low and high immunohistochemical staining for EZH2. (D) Kaplan-Meier curves of disease-specific mortality of patients whose ovarian vasculature expressed low versus high EZH2 (EZH2-Endo). EZH2-Endo was predictive of poor overall survival. (E) Representative images of human ovarian tumors with low or high immunohistochemical staining for VEGF. (F) VEGF expression was strongly associated with high EZH2-Endo (□p<0.01). (G) Representative images of human ovarian tumors with low or high immunohistochemical staining for microvessel density (MVD). (H) High MVD counts in the tumor was significantly associated with high EZH2-Endo expression (□p<0.001). Pictures in panels A, C, and E were taken at original magnification ×200, and in panel g at original magnification ×200.

FIG. 10A-C. VEGF increases EZH2 in endothelial cells. (A, B) Results are in response to 6-hour treatments with EGF [25 ng/μL], VEGF [50 ng/μL], conditioned medium (CM) from the non-cancerous ovarian epithelial cell line IOSE120, two ovarian cancer cell lines OVCA420 and SKOV3, and complete medium with either 10% serum (A) or 2% serum (B). Percent fold changes represent the mean+/−s.d. of triplicate experiments compared to untreated control cells. *p<0.05; **p<0.01; ***p<0.001. (A) EZH2 promoter activity is increased in an endothelial cell line in response to EGF, VEGF, and conditioned media from ovarian cancer cell lines. EAhy926 hybridoma endothelial cell line was cotransfected with the Renilla luciferase plasmid and firefly luciferase plasmid either with or without the EZH2 promoter construct followed by treatment with EGF, VEGF and conditioned medium and promoter activity was determined. (B) EZH2 mRNA levels are increased in HUVEC in response to EGF, VEGF, and conditioned media from ovarian cancer cell lines. Cells were treated as indicated and purified RNA was used in real-time quantitative RT-PCR. Control values were normalized using 3 housekeeping genes. (C) Pearson's analysis shows significant correlation between EZH2 and VEGF expression values (Log₂) from 29 microdissected high-grade serous papillary ovarian adenocarcinomas.

FIG. 11A-E. Physical characteristics of siRNA-chitosan nanoparticles. (A) Composition of CH/TPP/siRNA. (B) Mean particle size of siRNA-chitosan particles was measured using light scattering with a particle analyzer, showing that nanoparticles maintained 100-200 nm size up to 7:1 ratio (CH:TPP). (C) Zeta potential of siRNA-chitosan nanoparticles showed slight positive charge. (D) Incorporation efficiency of siRNA into chitosan nanoparticles with 3:1 ratio of CH:TPP resulting in >75% incorporation efficiency. (E) Atomic force microscopy (AFM) demonstrated that siRNA-chitosan nanoparticles were spherical and <150 nm in size.

FIG. 12A-C. Intracellular uptake of siRNA-chitosan nanoparticles. Increased binding efficiency of siRNA-chitosan nanoparticles was noted compared to naked siRNA. (A) Fluorescence microscopy image of HeyA8 cells after incubating either with siRNA alone or with siRNA-chitosan nanoparticles at 4° C. for 20 minutes in PBS. (B) Flow cytometry analysis demonstrated that uptake efficiency of nanoparticles into cells was increased by 72-fold after incubating cells in PBS at 4° C. for 20 minutes. (C) Graphical representation of percentage of uptake of Alexa-555 siRNA by cells by flow cytometry analysis.

FIG. 13A-E. In vivo siRNA delivery using chitosan nanoparticles. Distribution of siRNA following single i.v. injection of Alexa-555 siRNA-chitosan nanoparticles in orthotopic HeyA8 tumor bearing nude mice. Fluorescent siRNA distribution in tumor tissue: (A) H & E, original magnification ×200 (left); tumor tissues were stained with anti-CD31 antibody to detect endothelial cells (right). (B) 50 μm sections were stained with Cytox Green and examined with confocal microscopy (original magnification ×400) (left); lateral view (right), photographs taken every 1 μm were stacked and examined from the lateral view. Nuclei were labeled and fluorescent siRNA was seen throughout the section. At all time points, punctated emissions of the siRNA were noted in perinuclear region of individual cells and siRNA was seen in >80% of fields examined. (C) Western blot of lysate from orthotopic tumors collected after 24, 48, 72 and 96 hours after a single injection of control siRNA/CH or human (EZH2 Hs siRNA/CH). (D) EZH2 gene silencing in HeyA8 tumor as well as tumor endothelial cells. Tumors collected after 48 hours of single injection of control siRNA/CH, or EZH2 Hs siRNA/CH, or EZH2 Mm siRNA/CH and stained for EZH2 and CD31. Pictures were taken at original magnification, ×200. (E) Effects of EZH2 Hs siRNA/CH or EZH2 Mm siRNA/CH on tumor weight in mouse orthotopic tumor models. Nude mice were injected with HeyA8 or SKOV3ip1 ovarian cancer cells and 1 week later, were randomly assigned (10 mice per group) to receive therapy: (1) control siRNA/CH, (2) EZH2 Hs siRNA/CH, (3) EZH2 Mm siRNA/CH, and (4) combination of EZH2 Hs siRNA/CH plus EZH2 Mm siRNA/CH. Mice were sacrificed when any animals in control or a treatment group became moribund (after 3-4 weeks of therapy) and mouse weight, tumor weight and tumor location were recorded. Error bars represent s.e.m. *p<0.05; **p<0.001.

FIG. 14A-C. (A) Effect of tumor (EZH2 Hs siRNA/CH) or endothelial (EZH2 Mm siRNA/CH) targeted EZH2 siRNA on MVD and pericyte coverage. Tumors harvested following 3-4 weeks of therapy were stained for CD31 (MVD; red) and desmin (pericyte coverage; green). All pictures were taken at original magnification ×200. The bars in the graphs correspond sequentially to the labeled columns of images at left. Error bars represent s.e.m. *p<0.05; **p<0.001. (B) ChIP assay of EZH2 binding to human VASH1 promoter in response to VEGF in HUVEC. Cross-linked chromatin from HUVEC was treated with (+) or without (−) VEGF and immunoprecipitated (IP) using EZH2 or mouse IgG antibodies. The input and immunoprecipitated DNA were subjected to PCR using primers corresponding to the 3800 to 3584 base pairs upstream of VASH1 transcription start site. PCR products were examined on ethidium-bromide-stained agarose gel. (C) Effects of EZH2 gene silencing on VASH1 mRNA was analyzed using real-time qRT-PCR in MOEC. Fold difference in levels of VASH1 mRNA represents the mean of triplicate experiments compared to control siRNA treated cells. Error bars represent s.e.m. *p<0.01.

FIG. 15. Analysis of putative EZH2 pathways in epithelial tumor cell-associated endothelial cells. Pathway diagrams were generated with the assistance of Pathway Studio software (Ariadne, Rockville, Md.). VEGF stimulation of endothelial cells leads to increased expression of E2F3, which directly modulates EZH2 expression. EZH2, a transcriptional repressor, may have multiple targets, including anti-angiogenic, pro-apoptotic, and tumor suppressor genes.

FIG. 16A-B. Incorporation and stability of siRNA-chitosan nanoparticles. (A) Electrophoretic migration of naked siRNA and siRNA-chitosan nanoparticles. SiRNA-chitosan nanoparticles (open arrow) remained at top of the gel compared to naked siRNA (solid arrow), which migrated downward. (B) Electrophoretic migration of siRNA-chitosan nanoparticles in the presence of 50% serum. SiRNA-chitosan nanoparticles were collected at different time points of incubation at 37° C. (Lane 1; naked siRNA, Lanes 2 to 5; siRNA-chitosan nanoparticles). Naked siRNA (solid arrow) was degraded over 12 to 24 hours in serum containing media; whereas chitosan nanoparticles (open arrow) protected the siRNA from degradation in serum.

FIG. 17. Alexa-555 siRNA uptake into macrophages. Tumor tissues were collected after single injection of untagged control siRNA/CH or Alexa-555 siRNA/CH nanoparticles and stained with anti-f4/80 antibody to detect scavenging macrophages. Macrophages are seen surrounding nests of tumor cells and have minimal siRNA uptake. Left panel demonstrates lack of natural autofluorescence following injection of untagged control siRNA/CH. Pictures were taken at original magnification ×200 (left and middle) and ×400 (right).

FIG. 18. In vivo siRNA distribution to major organs. Histological sections from the liver, kidney, lung, brain, and heart tissues were collected after intravenous injection of 5 Alexa-555 siRNA/CH nanoparticles and exposed to hematoxylin and eosin (H&E) and Hoechst staining. Left panel represents H&E staining, middle panel represents natural auto-fluorescence of each tissue after a single injection of untagged control siRNA/CH and right panel denotes Alexa-555 siRNA/CH. All pictures were taken at original magnification ×200.

FIG. 19. Western blot of lysate collected 72 hours after transfection of HeyA8 cells or MOEC with control, human EZH2, or mouse EZH2 siRNA.

FIG. 20. Weight distribution of HeyA8 and SKOV3ip1 tumors. Seven days following tumor cell injection, mice were randomly divided into 4 groups (10 mice per group) to receive therapy: (1) control siRNA/CH, (2) EZH2 Hs siRNA/CH, (3) EZH2 Mm siRNA/CH, and (4) combination of EZH2 Hs siRNA/CH plus EZH2 Mm siRNA/CH. Mice were sacrificed when any animals in control or a treatment group became moribund (after 3 to 4 weeks of therapy) and tumor weight was recorded.

FIG. 21. Effects of EZH2 Hs siRNA/CH or EZH2 Mm siRNA/CH on proliferation. Tumors harvested following 3-4 weeks of therapy were stained for proliferating cell nuclear antigen (PCNA). All pictures were taken at original magnification ×100. The bars in the graphs correspond sequentially to the labeled columns of images at left. Error bars represent s.e.m. *p<0.05.

FIG. 22. EZH2 gene silencing in MOEC. Cells were transfected with control or mouse EZH2 siRNA and harvested after 72 hours. RNA was isolated and subjected to real-time quantitative RT-PCR. The fold difference in levels of EZH2 mRNA represents the mean of triplicate experiments compared to control siRNA treated cells. Error bars represent s.e.m. *p<0.05.

### DESCRIPTION OF ILLUSTRATIVE EMBODIMENTS

The present invention is in part based on the inventors' finding that drug delivery particles which include a chitosan and a polyphosphate can be applied in effective delivery of therapeutic agents or diagnostic agents. For example, the inventors have found that nanoparticles that are composed of chitosan and tripolyphosphate anion can be applied in the successful delivery of siRNA into tissues of a subject. The present invention is also in part based on the finding that nanoparticles which include a chitosan and an inhibitor of EZH2 have therapeutic application in the treatment of ovarian cancer.

### A. Chitosan and Analogs Thereof

The nanoparticles of the present invention include chitosan as a component. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum et al., 1991). The chitosan has a positive charge, stemming from the deacetylated amino group (—NH₃⁺). Chitosan, chitosan derivatives or salts (e.g., nitrate, phosphate, sulphate, hydrochloride, glutamate, lactate or acetate salts) of chitosan may be used and are included within the meaning of the term “chitosan.” As used herein, the term “chitosan derivatives” is intended to include ester, ether, or other derivatives formed by bonding of acyl and/or alkyl groups with —OH groups, but not the NH₂ groups, of chitosan. Examples are O-alkyl ethers of chitosan and O-acyl esters of chitosan. Modified chitosans, particularly those conjugated to polyethylene glycol, are also considered “chitosan derivatives.” Many chitosans and their salts and derivatives are commercially available (e.g., SigmaAldrich, Milwaukee, Wis.).

Methods of preparing chitosans and their derivatives and salts are also know, such as boiling chitin in concentrated alkali (50% w/v) for several hours—this produces chitosan wherein 70-75% of the N-acetyl groups have been removed. A non-limiting example of a chitosan, wherein all of the N-acetyl groups have been removed, is shown in formula (III) below.

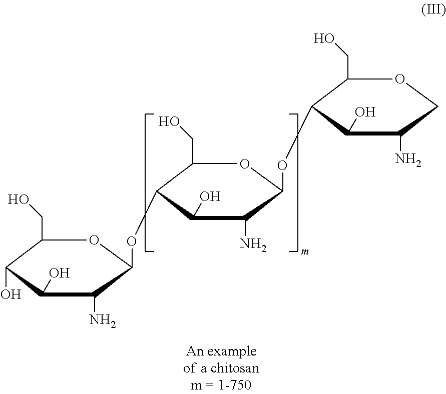

Chitosans may be obtained from any source known to those of ordinary skill in the art. For example, chitosans may be obtained from commercial sources. Chitosans may be obtained from chitin, the second most abundant biopolymer in nature. Chitosan is prepared by N-deacetylation of chitin. Chitosan is commercially available in a wide variety of molecular weight (e.g., 10-1000 kDa) and usually has a degree of deacetylation ranging between 70%-90%.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different molecular weights can be prepared by enzymatic degradation of high molecular weight chitosan using chitosanase or by the addition of nitrous acid. Both procedures are well known to those skilled in the art and are described in various publications (Li et al., 1995; Allan and Peyron, 1995; Domard and Cartier, 1989). The chitosan is water-soluble and may be produced from chitin by deacetylation to a degree of greater than 40%, preferably between 50% and 98%, and more preferably between 70% and 90%.

Some methods of producing chitosan involve recovery from microbial biomass, such as the methods taught by U.S. Pat. No. 4,806,474 and U.S. Patent Application No. 20050042735, herein incorporated by reference. Another method, taught by U.S. Pat. No. 4,282,351, teaches only how to create a chitosan-beta-glucan complex.

Chitosan derivatives are also suitable for use in this invention. Suitable chitosan derivatives include, without limitation, esters, ethers or other derivatives formed by bonding acyl and/or alkyl groups with the hydroxyl groups, but not the amino groups of chitosan. Examples include O-alkyl ethers of chitosan and O-acyl esters of chitosan.

The chitosan, chitosan derivative or salt used in the present invention is water soluble. Chitosan glutamate is water soluble. By “water soluble” we mean that that the chitosan, chitosan derivative or salt dissolves in water at an amount of at least 10 mg/ml at room temperature and atmospheric pressure. The chitosan, chitosan derivative, or salt used in the present invention have a positive charge. The positive charge is needed to prepare the particles by electrostatic interaction to negative charged materials such as phosphate ion, RNA, and DNA.

Additional information regarding chitosan and chitosan derivatives can be found in U.S. Patent App. Pub. Nos. 20070167400, 20070116767, 20070311468, 20060277632, 20060189573, 20060094666, 20050245482, 20050226938, 20040247632, and 20030129730, each of which is herein specifically incorporated by reference.

### B. Polyphosphates

Polyphosphates are phosphate polymers linked between hydroxyl groups and hydrogen atoms. A polyphosphate anion as used herein refers to a compound of formula (I):

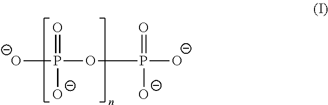

wherein n is an integer ranging from 2-10.

A “polyphosphate” as used herein refers to a compound of formula (II):

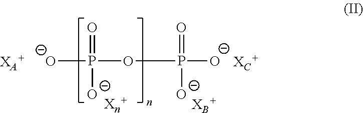

wherein n is an integer ranging from 2-10; and X_{A}, X_{B}, X_{C} and Xₙ are each independently any monovalent cation (e.g., H⁺, Na⁺, K⁺, Cs⁺, NH₄⁺).

In particular embodiments of the present invention, n=3, and the polyphosphate is a tripolyphosphate. In more particular embodiments, n=3 and X is Na⁺, and the phosphosphate is sodium tripolyphosphate.

In particular embodiments of the present invention, sodium tripolyphosphate is utilized in the nanoparticles and methods set forth herein. Sodium tripolyphosphate (STPP, pentasodium triphosphate, or sodium triphosphate), with formula Na₅P₃O₁₀, is a polyphosphate of sodium. It is the sodium salt of triphosphoric acid. Tripolyphosphates have a wide variety of applications, including as automatic dishwasher detergents, laundry detergents, cleaners, ceramics, food and beverages.

Tripolyphosphates can be obtained from natural or commercial sources, or can be chemically synthesized. Information regarding the synthesis of sodium tripolyphosphate can be found in U.S. Patent App. Pub. No. 20020170849, herein specifically incorporated by reference.

### C. Methods of Making Nanoparticles Comprising Chitosan and TPP

### 1. Preparation of a Chitosan Solution

The preferred process for preparing the nanoparticles of the invention is by mixing together the ingredients. Examples are set forth in detail in the specification below. In this process, chitosan (such as a powder of chitosan or a derivative thereof or a salt of chitosan or a salt of a derivative of chitosan) is dissolved in a suitable solvent to form a solution. For example, the solvent may be water, acetic acid, or hydrochloric acid.

The chitosan-containing solution that is formed may optionally be centrifuged to remove contaminants, although removal of all contaminants is not required.

The pH of the chitosan solution may then be adjusted such that the pH is in a range of about 3.5 to about 5.5. In more particular embodiments, the pH of the chitosan solution is adjusted so that it is in the range of about 4.0 to about 5.0. In still further particular embodiments, the pH of the chitosan solution is adjusted so that it is in the range of about 4.4 to about 4. In a particular embodiment, the pH of the chitosan solution is adjusted such that the pH is about 4.6. The pH can be adjusted by any method known to those of ordinary skill in the art. For example, the pH may be adjusted by the addition of NaOH, such as 10 N NaOH.

One or more additional components can optionally be added to the chitosan solution. Examples of such components include a therapeutic or diagnostic agent, such as any of those agents discussed below.

### 2. Preparation of a Polyphosphate Solution

A solution of polyphosphate is prepared by dissolving the polyphosphate in distilled water. The concentration of polyphosphate in the solution can be in the range of 0.01% to 1.0%.

In particular embodiments, the polyphosphate is a tripolyphosphate (TPP). The solvent may be any solvent, such as any of those solvents set forth elsewhere in this specification. For example, the concentration of TPP in the solution may be about 0.01% to about 1.00%. In more particular embodiments, the concentration is about 0.1% to about 0.9%. In more particular embodiments, the concentration is about 0.1% to about 0.5%. In even more particular embodiments, the concentration is about 0.2% to about 0.3%. In a particular embodiment, the concentration of TPP is about 0.25%.

In some embodiments, a therapeutic or diagnostic agent is added to the polyphosphate solution. In some embodiments, a therapeutic or diagnostic agent is added to the polyphosphate solution. For example, the agent may be a therapeutic agent, such as siRNA.

### 3. Mixing of the Chitosan Solution and the Polyphosphate Solution

The chitosan solution is then added to the polyphosphate solution. As discussed above, the polyphosphate solution optionally includes one or more therapeutic or diagnostic agents.

In particular embodiments, the mixture is allowed to incubate at 4° C. for a period of time, such as one hour. This step assists with stabilization of the particles.

Mixing of the chitosan solution and the polyphosphate solution results in the formation of nanoparticles. The nanoparticles are composed of chitosan, polyphosphate, and any therapeutic or diagnostic agent(s) that were included.

### 4. Purification

The nanoparticles can be purified using any method known to those of ordinary skill in the art. In particular embodiments, the nanoparticles may be purified by centrifugation and removal of supernatant. For example, centrifugation may be at 12000 rpm for about 30 min to about 60 min. Centrifugation may be repeated once, or more than once. In particular embodiments, centrifugation is repeated three times.

### 5. Analysis of Formed Nanoparticles

Nanoparticles that are formed by the present methods can be analyzed using any method and technique known to those of ordinary skill in the art. For example, particle size may be measured by dynamic light scattering.

The nanoparticles that are formed can be of any size. For example, the particles may be of a size in the range of about 10 nm to about 1000 nm in size or greater. In some embodiments, the particles are of a size in the range of about 1 μm to 1000 μm in size.

In some embodiments, particle size is heterogeneous and poorly defined. If desired, particle size may be reduced using any method known to those of ordinary skill in the art. The particle size can be controlled using standard techniques such as sieving.

### 6. Storage

The nanoparticles may be stored using any method known to those of ordinary skill in the art. The nanoparticles may be stored at 4° C. until ready for use.

### 7. Optional Ingredients

The particles of the present invention may optionally include one or more additional ingredients. Examples of additional ingredients include, but are not limited to, sugars such as sucrose and trehalose; polyols such as mannitol and sorbitol; and surfactants such as polysorbates; amino acids such as glycine; and polyethylene glycol. The total amount of additional ingredients may be up to a total of about 10% by weight of the nanoparticle.

### D. Therapeutic and Diagnostic Agents

A “therapeutic agent” as used herein refers to any agent that can be administered to a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

A “diagnostic agent” as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA. In particular embodiments, the therapeutic or diagnostic agent is a siRNA. siRNA is discussed in greater detail in the specification below.

The therapeutic agent or diagnostic agent can be any such agent known to those of ordinary skill in the art. For example, the therapeutic agent may be an anti-inflammatory agent, an anti-infective agent, an agent that can be applied in the treatment of a hyperproliferative disease such as cancer, an agent that can be applied in the treatment of a degenerative disease, and so forth.

Other examples of therapeutic agents include, but are not limited to, agents for the prevention of restenosis, agents for treating renal disease, agents used for intermittent claudication, agents used in the treatment of hypotension and shock, angiotensin converting enzyme inhibitors, antianginal agents, anti-arrhythmics, anti-hypertensive agents, antiotensin ii receptor antagonists, antiplatelet drugs, b-blockers b 1 selective, beta blocking agents, botanical product for cardiovascular indication, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators, potassium channel openers, potassium salts, anticonvulsants, antiemetics, antinauseants, anti-parkinson agents, antispasticity agents, cerebral stimulants, agents that can be applied in the treatment of trauma, agents that can be applied in the treatment of Alzheimer disease or dementia, agents that can be applied in the treatment of migraine, agents that can be applied in the treatment of neurodegenerative diseases, agents that can be applied in the treatment of kaposi's sarcoma, agents that can be applied in the treatment of AIDS, cancer chemotherapeutic agents, agents that can be applied in the treatment of immune disorders, agents that can be applied in the treatment of psychiatric disorders, analgesics, epidural and intrathecal anesthetic agents, general, local, regional neuromuscular blocking agents sedatives, preanesthetic adrenal/acth, anabolic steroids, agents that can be applied in the treatment of diabetes, dopamine agonists, growth hormone and analogs, hyperglycemic agents, hypoglycemic agents, oral insulins, largevolume parenterals (lvps), lipid-altering agents, metabolic studies and inborn errors of metabolism, nutrients/amino acids, nutritional lvps, obesity drugs (anorectics), somatostatin, thyroid agents, vasopressin, vitamins, corticosteroids, mucolytic agents, pulmonary anti-inflammatory agents, pulmonary surfactants, antacids, anticholinergics, antidiarrheals, antiemetics, cholelitholytic agents, inflammatory bowel disease agents, irritable bowel syndrome agents, liver agents, metal chelators, miscellaneous gastric secretory agents, pancreatitis agents, pancreatic enzymes, prostaglandins, prostaglandins, proton pump inhibitors, sclerosing agents, sucralfate, anti-progestins, contraceptives, oral contraceptives, not oral dopamine agonists, estrogens, gonadotropins, GNRH agonists, GHRH antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors, metal chelators, prostaglandins, vitamin K, anti-androgens, aminoglycosides, antibacterial agents, sulfonamides, cephalosporins, clindamycins, dermatologics, detergents, erythromycins, anthelmintic agents, antifungal agents, antimalarials, antimycobacterial agents, antiparasitic agents, antiprotozoal agents, antitrichomonads, antituberculosis agents, immunomodulators, immunostimulatory agents, macrolides, antiparasitic agents, corticosteroids, cyclooxygenase inhibitors, enzyme blockers, immunomodulators for rheumatic diseases, metalloproteinase inhibitors, nonsteroidal anti-inflammatory agents, analgesics, antipyretics, alpha adrenergic agonists/blockers, antibiotics, antivirals, beta adrenergic blockers, carbonic anhydrase inhibitors, corticosteroids, immune system regulators, mast cell inhibitors, nonsteroidal anti-inflammatory agents, prostaglandins, and proteolytic enzymes.

Examples of diagnostic agents include, but are not limited to, magnetic resonance image enhancement agents, positron emission tomography products, radioactive diagnostic agents, radioactive therapeutic agents, radio-opaque contrast agents, radiopharmaceuticals, ultrasound imaging agents, and angiographic diagnostic agents.

In particular embodiments, the therapeutic agent is a chemotherapeutic agent. A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegaI1; dynemicin, including dynemicin A); bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In particular embodiments, as discussed above, the therapeutic agent is a siRNA. Examples of such siRNA are discussed in greater detail below.

### E. Inhibition of Gene Expression and siRNA

siNA (e.g., siRNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90 to 100, 150, 200, 250, 300, 350, 400, 450, 500 or more contiguous nucleobases, including all ranges therein. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

Agents of the present invention useful for practicing the methods of the present invention include, but are not limited to siRNAs. Typically, introduction of double-stranded RNA (dsRNA), which may alternatively be referred to herein as small interfering RNA (siRNA), induces potent and specific gene silencing, a phenomena called RNA interference or RNAi. This phenomenon has been extensively documented in the nematode C. elegans (Fire et al., 1998), but is widespread in other organisms, ranging from trypanosomes to mouse. Depending on the organism being discussed, RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80, 85, 90, 95, 98,% or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides, and are able to modulate the gene expression. In the context of the present invention, the siRNA is preferably less than 500, 200, 100, 50 or 25 nucleotides in length. More preferably, the siRNA is from about 19 nucleotides to about 25 nucleotides in length.

A target gene generally means a polynucleotide comprising a region that encodes a polypeptide, or a polynucleotide region that regulates replication, transcription or translation or other processes important to expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome, or it may be present on an extrachromosomal genetic construct such as a plasmid or a cosmid. The targeted gene can also be derived from a pathogen, such as a virus, bacterium, fungus or protozoan, which is capable of infecting an organism or cell. Target genes may be viral and pro-viral genes that do not elicit the interferon response, such as retroviral genes. The target gene may be a protein-coding gene or a non-protein coding gene, such as a gene which codes for ribosomal RNAs, splicosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present invention to modulate or attenuate target gene expression: developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), tumor suppressor genes (e.g., APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide), pro-apoptotic genes (e.g., CD95, caspase-3, Bax, Bag-1, CRADD, TSSC3, bax, hid, Bak, MKP-7, PARP, bad, bcl-2, MST1, bbc3, Sax, BIK, and BID), cytokines (e.g., GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, PDGF, and mda7), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), and enzymes (e.g., ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, pectinesterases, peroxidases, phosphatases, phospholipases, phophorylases, polygalacturonases, proteinases and peptideases, pullanases, recombinases, reverse transcriptases, topoisomerases, xylanases).

siRNA can be obtained from commercial sources, natural sources, or can be synthesized using any of a number of techniques well-known to those of ordinary skill in the art. For example, one commercial source of predesigned siRNA is Ambion®, Austin, Tex. Another is Qiagen® (Valencia, Calif.). An inhibitory nucleic acid that can be applied in the compositions and methods of the present invention may be any nucleic acid sequence that has been found by any source to be a validated downregulator of a protein of interest. For example, in a particular embodiment, the inhibitory nucleic acid is Qiagen® (Valencia, Calif.) validated siRNA product Catalog Number SIO2662338.

In one aspect, the invention generally features an isolated siRNA molecule of at least 19 nucleotides, having at least one strand that is substantially complementary to at least ten but no more than thirty consecutive nucleotides of a nucleic acid that encodes an EZH2 protein, and that reduces the expression of the EZH2 protein. In a particular embodiment of the present invention, the siRNA molecule has at least one strand that is substantially complementary to at least ten but no more than thirty consecutive nucleotides of the mRNA that encodes EZH2.

In another particular embodiment, the siRNA molecule is at least 75, 80, 85, or 90% homologous, preferably 95%, 99%, or 100% homologous, to at least 10 contiguous nucleotides of any of the nucleic acid sequences encoding a full-length EZH2 protein, such as GenBank Accession number 152998 (SEQ ID NO:1) and GenBank Accession number 004456 (SEQ ID NO:2). Without undue experimentation and using the disclosure of this invention, it is understood that additional siRNAs can be designed and used to practice the methods of the invention.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 20040019001 and U.S. Pat. No. 6,673,611 (each of which is incorporated by reference in its entirety). Collectively, all such altered nucleic acids or RNAs described above are referred to as modified siRNAs.

Preferably, RNAi is capable of decreasing the expression of a protein, such as EZH2, by at least 10%, 20%, 30%, or 40%, more preferably by at least 50%, 60%, or 70%, and most preferably by at least 75%, 80%, 90%, 95% or more.

Certain embodiments of the present invention pertain to methods of inhibiting expression of a gene encoding a protein in a cell. In a specific embodiment, the protein is EZH2. Introduction of siRNA into cells can be achieved by methods known in the art, including for example, microinjection, electroporation, or transfection of a vector comprising a nucleic acid from which the siRNA can be transcribed. Alternatively, a siRNA can be directly introduced into a cell in a form that is capable of binding to target mRNA transcripts. To increase durability and membrane-permeability the siRNA may be combined or modified with liposomes, poly-L-lysine, lipids, cholesterol, lipofectine or derivatives thereof. In certain aspects cholesterol-conjugated siRNA can be used (see, Song et al., 2003).

### F. Nucleic Acids

The present invention provides methods and compositions for the delivery of siNA via neutral liposomes. Because a siNA is composed of a nucleic acid, methods relating to nucleic acids (e.g., production of a nucleic acid, modification of a nucleic acid, etc.) may also be used with regard to a siNA.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may formed by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “ss” and a double stranded nucleic acid by the prefix “ds”.

### 1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and

T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, carboxyalkyl, etc.) moeities comprise of from about 1, about 2, about 3, about 4, about 5, to about 6 carbon atoms. A nucleobase may be comprised in a nucleoside or nucleotide, using any chemical or natural synthesis method described herein or known to one of ordinary skill in the art.

### 2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

### 3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

### 4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides, or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-0 position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

### 5. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR™, to reduce false positives and discriminate between single base mutants, as described in U.S. Pat. No. 5,891,625. Other modifications and uses of nucleic acid analogs are known in the art, and it is anticipated that these techniques and types of nucleic acid analogs may be used with the present invention. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

### 6. Preparation of Nucleic Acids

A nucleic acid may be made by any technique known to one of ordinary skill in the art, such as chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

### 7. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain embodiments, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

### 8. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

### G. Treatment and Prevention of Disease

### 1. Definitions

“Treatment” and “treating” as used herein refer to administration or application of a therapeutic agent to a subject or performance of a procedure or modality on a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, reduction in the size of a tumor.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. For example, a subject at risk of developing cancer may be administered an effective amount of a composition comprising nanoparticles of the present invention to reduce the risk of development of the cancer compared to the risk in a subject that did not receive nanoparticles.

“Determining prognosis” as used herein refers to predicting the likelihood that a subject with have a certain course or outcome of a disease. For example, in some embodiments determining prognosis involves determining likelihood of reduced survival or likelihood of tumor growth.

### 2. Diseases to be Treated or Prevented

Certain embodiments of the present invention concern methods of treating or preventing disease in a subject involving administration of nanoparticles of the present invention. The disease may be any disease that can affect a subject. For example, the disease may be a hyperproliferative disease, an inflammatory disease, or an infectious disease. In particular embodiments, the disease is a hyperproliferative disease. In more particular embodiments, the disease is cancer.

The cancer can be any cancer. For example, the cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

### H. Pharmaceutical Preparations

Certain of the methods set forth herein pertain to methods involving the administration of a pharmaceutically effective amount of a composition comprising nanoparticles of the present invention.

### 1. Compositions

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present invention may comprise different types of carriers depending on whether it is to be administered in solid, liquid or aerosol form, and whether it need to be sterile for such routes of administration as injection.

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations should meet sterility, pyrogenicity, general safety and purity standards as required by FDA Office of Biologics standards.

The compositions comprising nanoparticles may be extensively dialyzed to remove undesired small molecular weight molecules and/or lyophilized for more ready formulation into a desired vehicle, where appropriate. The active compounds will then generally be formulated for administration by any known route, such as parenteral administration. Methods of administration are discussed in greater detail below.

The present invention contemplates methods using compositions that are sterile solutions for intravascular injection or for application by any other route as discussed in greater detail below. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

The formulation of the composition may vary depending upon the route of administration. For parenteral administration in an aqueous solution, for example, the solution should be suitably buffered if necessary and the liquid diluent first rendered isotonic with sufficient saline or glucose. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, formulations for administration via an implantable drug delivery device, and any other form. One may also use nasal solutions or sprays, aerosols or inhalants in the present invention.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

In certain embodiments, pharmaceutical composition includes at least about 0.1% by weight of the active agent. The composition may include, for example, about 0.01% In other embodiments, the pharmaceutical composition includes about 2% to about 75% of the weight of the composition, or between about 25% to about 60% by weight of the composition, for example, and any range derivable therein.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition must be stable under the conditions of manufacture and storage, and preserved against the contaminating action of microorganisms, such as bacteria and fungi. It will be appreciated that exotoxin contamination should be kept minimally at a safe level, for example, less that 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, one may use nasal solutions or sprays, aerosols or inhalants in the present invention. Nasal solutions are usually aqueous solutions designed to be administered to the nasal passages in drops or sprays.

Sterile injectable solutions are prepared by incorporating the nanoparticles in the required amount in the appropriate solvent with various of the other ingredients enumerated above, as required, followed by sterilization.

### 2. Routes of Administration

Upon formulation, nanoparticles will be administered in a manner compatible with the dosage formulation and in such amount as is therapeutically effective.

The nanoparticles can be administered to the subject using any method known to those of ordinary skill in the art. For example, a pharmaceutically effective amount of a composition comprising nanoparticles may be administered intravenously, intracerebrally, intracranially, intrathecally, into the substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990). In particular embodiments, the composition is administered to a subject using a drug delivery device.

### 3. Dosage

A pharmaceutically effective amount of the nanoparticles is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent. The administration could be intra-operative or post-operative.

### I. Combination Treatments

Certain embodiments of the present invention provide for the administration or application of one or more secondary forms of therapies for the treatment or prevention of a disease. For example, the disease may be a hyperproliferative disease, such as cancer.

The secondary form of therapy may be administration of one or more secondary pharmacological agents that can be applied in the treatment or prevention of cancer.

If the secondary therapy is a pharmacological agent, it may be administered prior to, concurrently, or following administration of the nanoparticles.

The interval between the administration of the nanoparticles and the secondary therapy may be any interval as determined by those of ordinary skill in the art. For example, the interval may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. In some embodiments, the timing of administration of a secondary therapeutic agent is determined based on the response of the subject to the nanoparticles.

Various combinations may be employed. For the example below an inhibitor of gene expression therapy is “A” and an anti-cancer therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

### 1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### J. Kits and Diagnostic Agents

In various aspects of the invention, a kit is envisioned containing nanoparticles or ingredients for the formation of nanoparticles of the present invention in one or more suitable container means. A suitable container means is a container that will not react with components of the kit, such as an eppendorf tube, an assay plate, a syringe, a bottle, or a tube. The container may be made from sterilizable materials such as plastic or glass. In some embodiments, the kit includes a composition comprising nanoparticles in one or more container means. In other embodiments, the kit includes a single container means that comprises chitosan or a solution comprising chitosan, and a separate container means that comprises a polyphosphate or a solution that comprises a polyphosphate, designed for admixture prior to use.

In some further embodiments, the kit includes one or more therapeutic or diagnostic agents. The one or more therapeutic or diagnostic agents may be in the same container means with the polyphosphate and/or chitosan.

The kit may further include an instruction sheet that outlines the procedural steps of the methods, and will follow substantially the same procedures as described herein or are known to those of ordinary skill.

### K. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Method for Preparation of Nanoparticles for the Delivery of siRNA

Chitosan nanoparticles were prepared according to the procedure based on the ionic gelation of chitosan with tripolyphosphate anions. The formation of the particles was a result of the interaction between the negatively charged groups of the tripolyphosphate and the positively charged amino groups of chitosan.

Chitosan with the deacetylation degree of 75-85% and the viscosity of 20-200 cP was dissolved at 0.5% (w/v) with 1% (v/v) acetic acid. The pH of the chitosan gel raised to 4.6 with 10 N NaOH. NaOH was added under magnetic stirring (high speed) drop by drop (each drop should be 4 pl) to raise pH. Chitosan nanoparticles formed spontaneously upon addition of aqueous tripolyphosphate solution to chitosan solution under magnetic stirring at 1200 rpm and mixed for further 10 minutes after addition of tripolyphosphate (Chitosan to TPP weight ratio is 6:1 and chitosan to TPP volume ratio is 3:1). The particles were then incubated at room temperature for 20 min. Nanoparticles were purified by centrifugation at 9000 g for 30 min at 5° C. Supernatants were discarded, and the chitosan nanoparticles were extensively rinsed with distilled water to remove any sodium hydroxide. Nanoparticles were resuspended in ultrapure water.

### SiRNA Entrapment in Chitosan Nanoparticles

For the association of siRNA with the chitosan-TPP nanoparticles, siRNA in buffer was added to the TPP solution before adding this drop-wise to the chitosan solution under constant magnetic stirring (1200 rpm) at room temperature and mixed for further 10 minutes after addition of tripolyphosphate. (Chitosan to siRNA weight ratio is 150:1 and chitosan to TPP weight ratio is 6:1, chitosan to TPP volume ratio is 3:1). The particles were then incubated at room temperature for 20 min. Nanoparticles were purified by centrifugation at 9000 g for 30 min at 5° C. Supernatants were discarded, and the chitosan nanoparticles were extensively rinsed with distilled water to remove any sodium hydroxide. Nanoparticles were resuspended in ultrapure water.

### Example 2

### Method of Preparation of Nanoparticles for the Delivery of siRNA

The following is another example of a method for the preparation of chitosan particles. About 50 milligrams of Chitosan (Mw. 50000-190000, Sigma) was dissolved in 10 ml of 0.25% acetic acid solution. Chitosan solution was isolated by centrifugation to remove contaminants. The pH of this mixture was adjusted to 4.6 with 10 N NaOH. 0.25% TPP (tripolyphosphate) solution was prepared. 140 ul of TPP (0.35 mg) and 35 ug of siRNA were mixed. Chitosan solution was added to 175 μl of both TPP and siRNA solution. The mixture was incubated in ice (4° C.) for 1 hr. The mixture was purified by centrifugation at 12000 rpm for 40 min at 4° C. (three times). After purification, chitosan nanoparticles were obtained.

TABLE 1
Formulation of siRNA-incorporated chitosan particles

| Weight ratio (chitosan:TPP) | Chitosan (mg) | TPP (mg) |
|---|---|---|
| 1:1 | 0.35 | 0.35 |
| 3:1 | 1.05 | 0.35 |
| 5:1 | 1.75 | 0.35 |
| 7:1 | 2.45 | 0.35 |
| 9:1 | 3.15 | 0.35 |
| 11:1 | 3.85 | 0.35 |
| 13:1 | 4.55 | 0.35 |
| 15:1 | 5.25 | 0.35 |

Total volume: 700 μl
Chitosan volume: 525 μl
TPP volume: 140 μl (0.35 mg)
siRNA volume: 35 μl (35 μg)

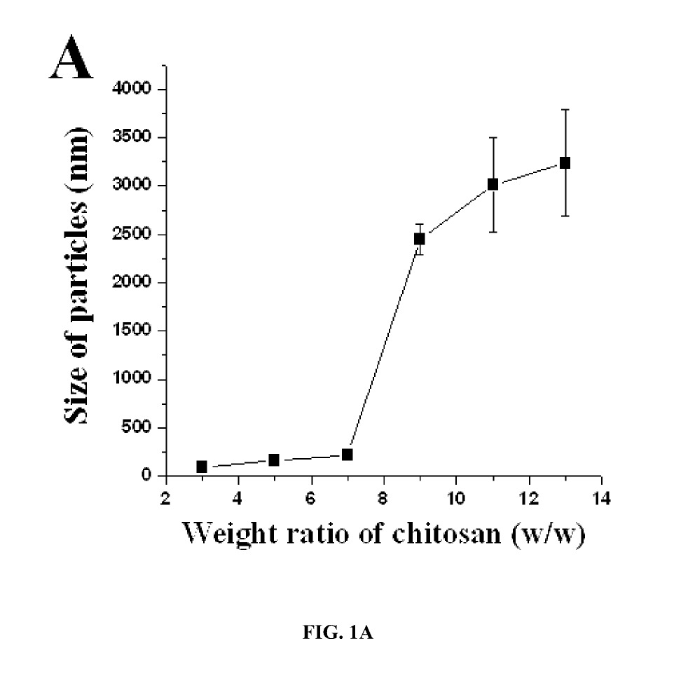

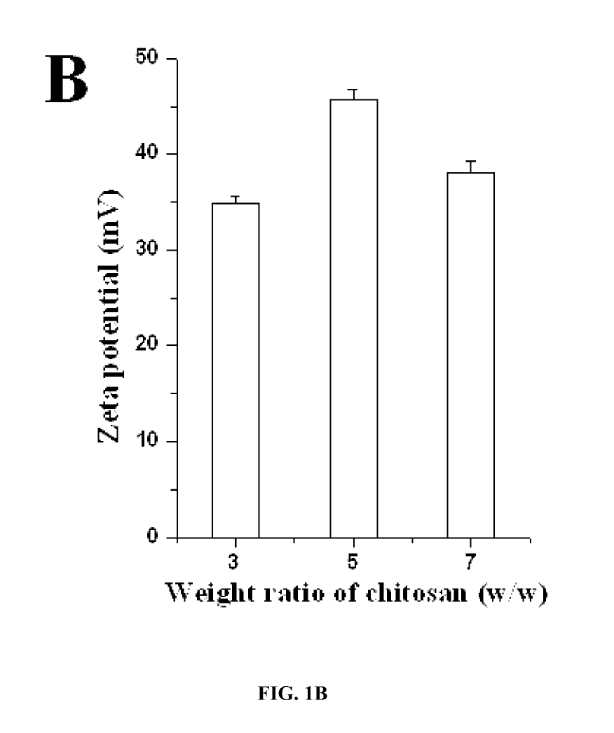

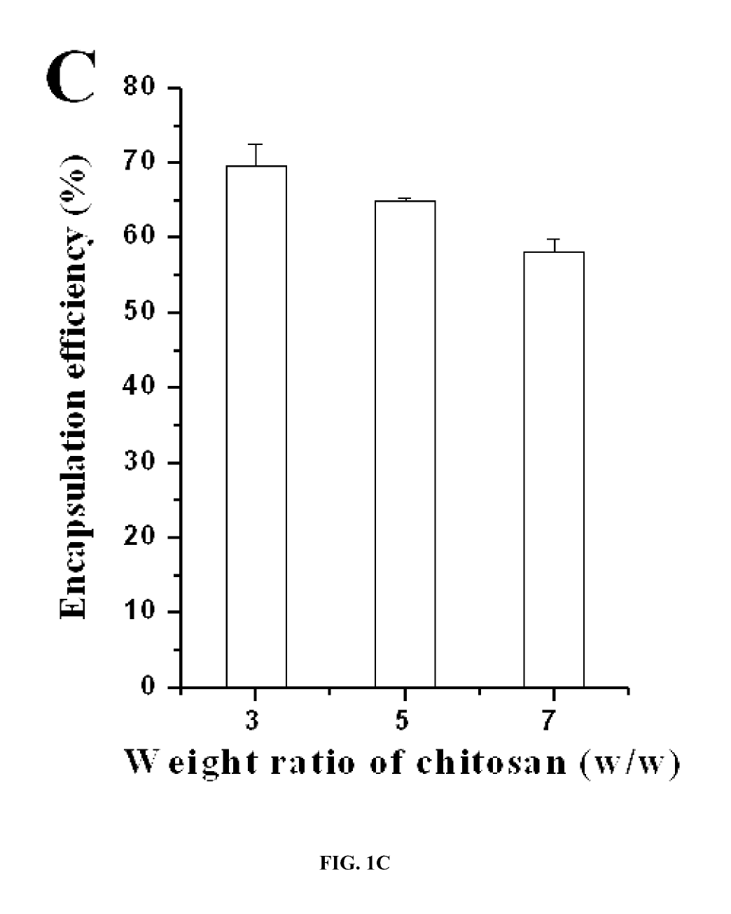

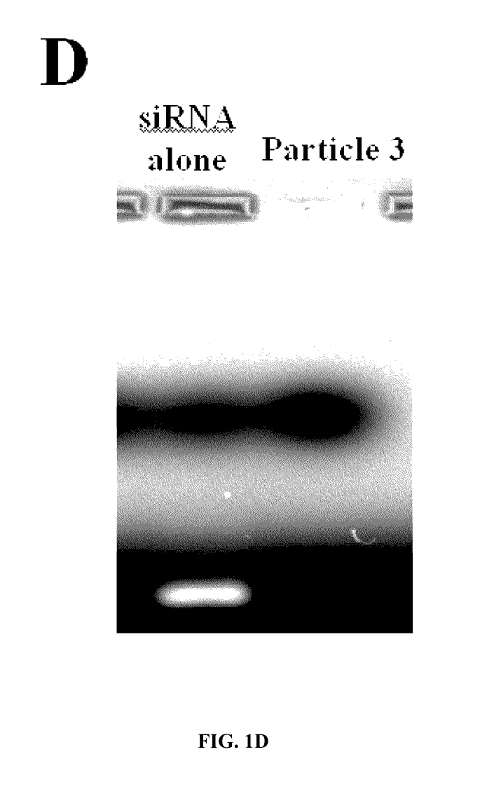

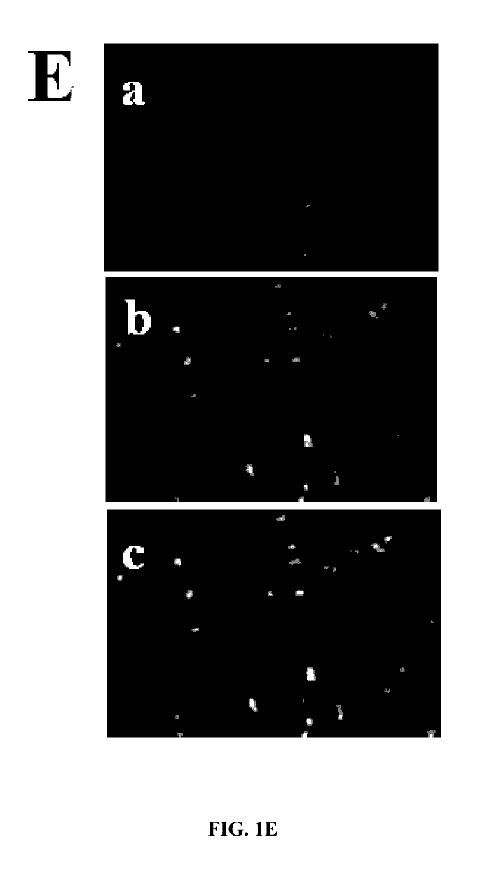

Results of studies assessing the physical properties of the nanoparticles are shown in FIG. 1. The siRNA-chitosan particles were prepared as following formulation in Table 1 and the physical properties of the various siRNA-chitosan particles were shown in FIG. 1. The mean particle size of the various siRNA-chitosan particles was approximately 100-3,200 nm (FIG. 1A). The particles size is dramatically increased up to weight ratio of 8:1 of chitosan respect with siRNA and TPP concentration, and in this formulation, it was observed that the siRNA-chitosan particles were slightly aggregated. In contrast, the particles size in the range of weight ratio of 3-7 chitosan is around 130 nm. Therefore, siRNA-CH3, CH5, and CH7 particles were accepted in further study. In addition, the zeta potential of siRNA-CH3, CH5, and CH7 particles showed positive charge especially 30-40 mV (FIG. 1B). The encapsulation efficiency of siRNA into chitosan particles shows in FIG. 1C. The siRNA-CH3 particles showed the highest encapsulation efficiency as compared to the other siRNA-CH5 and siRNA-CH7. Based on the results in FIGS. 1A, B, and C, the siRNA-CH3 was selected as the optimum formation of siRNA-chitosan particles and this formulation was accepted for the further in vitro and in vivo study. To observe the siRNA incorporation into particles, the electorophoresis analysis was performed. As shown in FIG. 1D, the band of siRNA-CH3 particle was stayed on the top of the gel caused by their positive charge. Moreover, the band is bright with EtBr staining, indicating that siRNA is incorporated into chitosan particles. On the other hand, the morphology of siRNA-chitosan particles is shown in FIG. 1E.

Encapsulation efficiency was next evaluated. After centrifugation (12000 rpm, 40 min) of alexa555 siRNA-incorporated chitosan particles, the supernatant was collected. The encapsulation efficiency was determined by fluorescence spectrophotometer measuring fluorescence intensity of supernatant.

Stability test in serum was next evaluated. 10 ul of 100% serum (FBS) and 10 ul of the particle were mixed and subsequently incubated for predetermined time at 37° C. 0.25 ug of siRNA and 5 ul of 100% serum were mixed and incubated for predetermined time at 37° C. Gel (4% agarose gel, 0.5 TAE) electrophoresis was performed by applying 100 V for 1 hr, and the bands were visualized by ethidium bromide staining. Stability test with PLLA (poly-L-aspartic acid) was next evaluated. Different concentration of PLLA were added to the particle to vary the weight ratio of PLLA:siRNA from 0 to 400. The reaction mixture was electrophoresed to determine whether RNA dissociated from the complex.

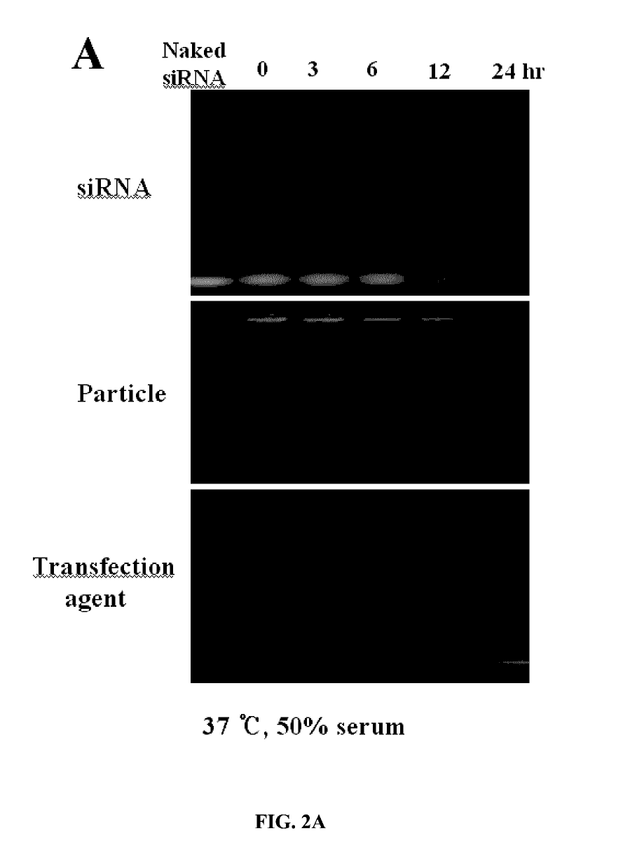

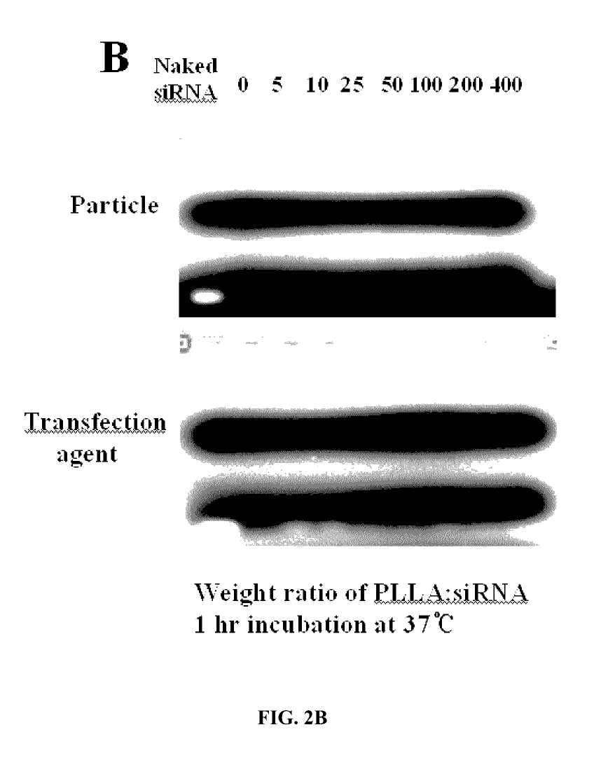

The influence of particles stability was examined by observing the electrophoretic migration behavior in the presence of serum and anionic material such as PLLA (FIG. 2). As shown in FIG. 2A, siRNA alone in 50% serum resulted in slightly degradation (loss of band in the gel) after several hours, but siRNA incorporated in the chitosan particles was not degraded, indicating that the chitosan particles protect siRNA degradation in serum. In serum, there are many anionic materials that would compete with and substitute for siRNA in the particles to confirm dissociation of siRNA from chitosan particles. Such a substitution has been assumed to be a major destabilization mechanism for the particles. Therefore, the stability of the siRNA-chitosan particles against PLLA as a model polyanion was performed. As shown in FIG. 2B, siRNA dissociated from the positive control, RNAipec®. In contrast, the siRNA-CH3 particles were stable up to a PLLA:siRNA weight ratio of 200. This result indicates that the interaction between chitosan particles and siRNA in the complex is much stronger than that of complex composed of commercialized product, RNAipet®, and siRNA.

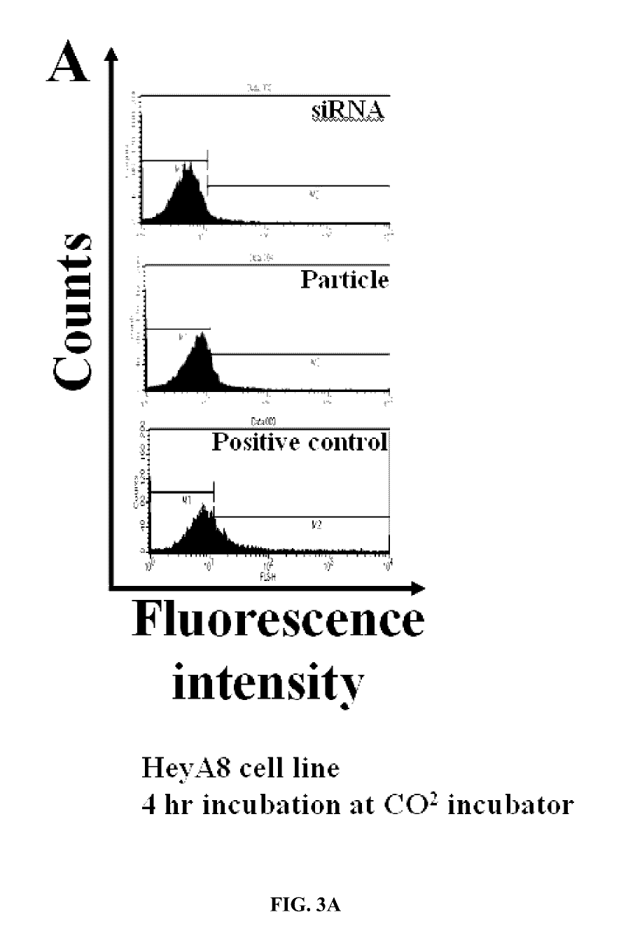

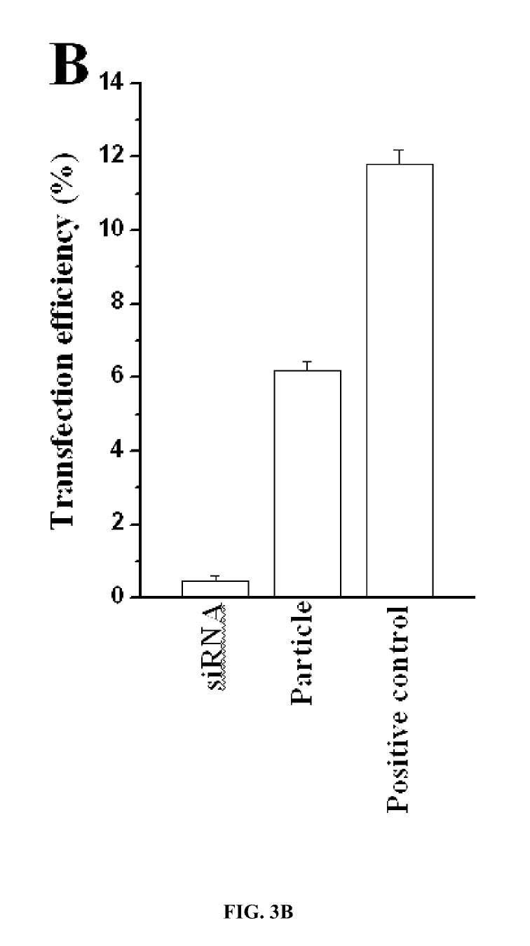

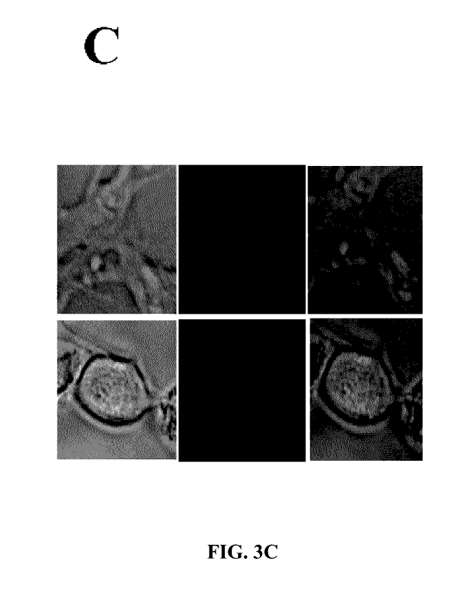

A transfection test was next performed. Cells (HeyA8) were seeded at 1×10⁵ cells in a six well plate one day before the transfection. After washing the cells with serum free media, 2 ml of RPMI1640 was added to each well in the case of the transfection performed without serum. One hundred microliters of the particle (5 ug of RNA basis) were prepared and added to the cells After 4 hr incubation at 37° C., the cells were washed with serum-free media to remove the remaining chitosan particles. The cells were fed again with RPMI1640 containing 10% FBS, cultured for 24 hr after transfection. After incubation, cells were observed by fluorescence microscopy or flow cytometry. Results are shown in FIG. 3. Results showed in vitro transfection study was performed to characterize the particles-mediated intracellular activity of the siRNA-CH3 particles (FIG. 3). As shown in FIG. 3A, siRNA-chitosan particles were transfected in HeyA8 cells as compared to siRNA alone. In addition, the transfection efficiency of siRNA-chitosan particles was 6-fold higher than that of siRNA alone (FIG. 3B). The chitosan-mediated particle system has many advantages because chitosan was composed cationic polysaccharide. Especially, the protonated amine groups of chitosan particles allow transport across cellular membranes and subsequent endocytosis into cells. Thus, the siRNA-chitosan particles, in this study, might be enhanced transfection efficiency caused by particle-mediated endocytosis mechanism. Based on this result, particle-mediated transfection was confirmed by taking a fluorescence microscopy image (FIG. 3C).

### Example 3

### Role of the Spinal M2 Receptor Subtype in the Analgesic Effect of the Muscarinic Receptor Agonist

The muscarinic acetylcholine receptors (mAChRs) in the spinal cord are important for the regulation of pain transmission. There are three mAChR subtypes in the spinal dorsal horn: M2, M3, and M4. However, the relative contribution of each mAChR subtype in the spinal cord to pain modulation remains unclear. Because the specificity of the available agonists and antagonists for each mAChR subtype is limited for in vivo use, it will be difficult to assess the contribution of individual mAChR subtypes to the spinal muscarinic analgesia by using pharmacological approaches.

Compared with the antisense technique, which requires potentially toxic concentrations to achieve gene-specific suppression, the efficient and reproducible silencing effects of double-stranded siRNA make RNA interference highly advantageous. Efficient delivery of siRNA into the neural tissues in vivo, however, has proven to be one of the major difficulties in the application of this novel technique. This example pertains to the identification of a very efficient way to deliver siRNA to the spinal cord in vivo is by using the chitosan-based siRNA nanoparticle delivery system of the present invention.

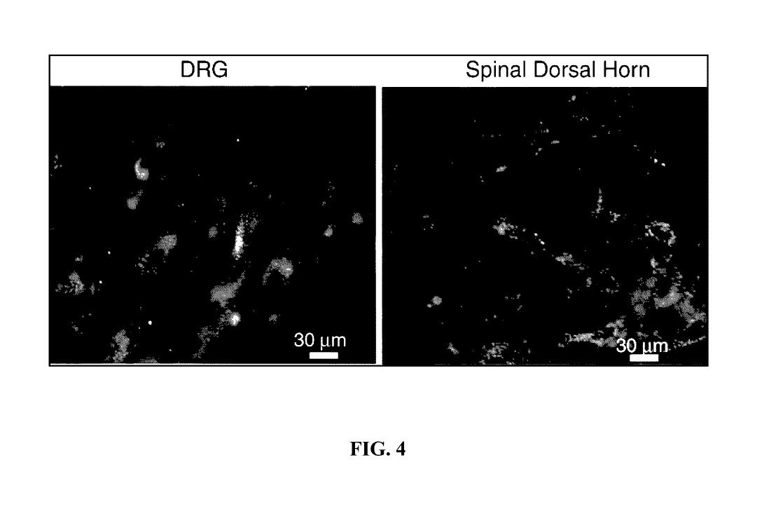

A study was conducted to evaluate the uptake and distribution of the siRNA-chitosan tagged with a fluorescent dye, Alexa-555, after intrathecal injection. An intrathecal catheter was implanted with its tip positioned to the lumbar spinal cord level in adult rats under isoflurane anesthesia. The siRNA fluorescence was present in the cytoplasm of neurons in the lumbar dorsal root ganglion (DRG) and the spinal cord within 24 hr after intrathecal injection of 5 μg of the tagged RNA encapsulated in chitosan (FIG. 4). However, when the fluorescence-tagged siRNA was injected without chitosan, its uptake in the DRG and spinal cord was negligible. Also, when the fluorescence-tagged siRNA was conjugated to the liposome 1,2-dioleoyl-sn-glycero-3-phosphatidylcholine (DOPC), there was no evidence of uptake by the DRG and spinal dorsal horn neurons upon intrathecal injection. These data demonstrate the effective uptake of siRNA by the sensory and spinal cord neurons using chitosan as a carrier.

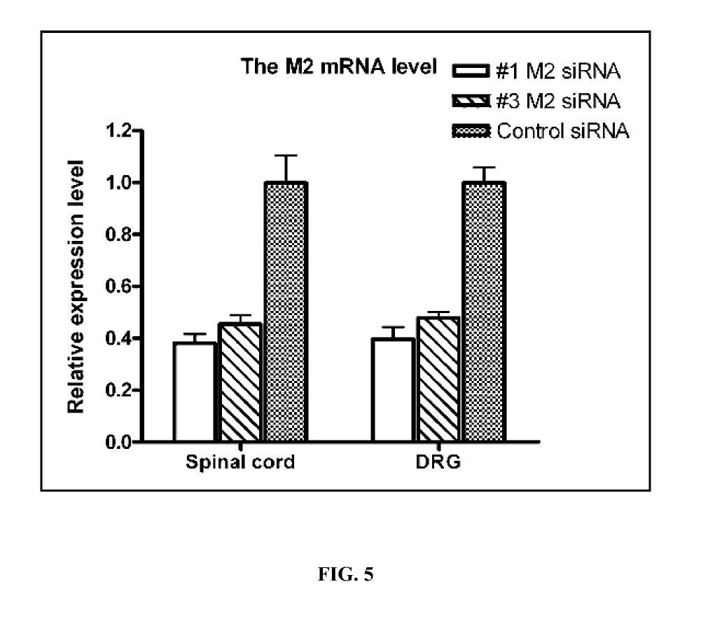

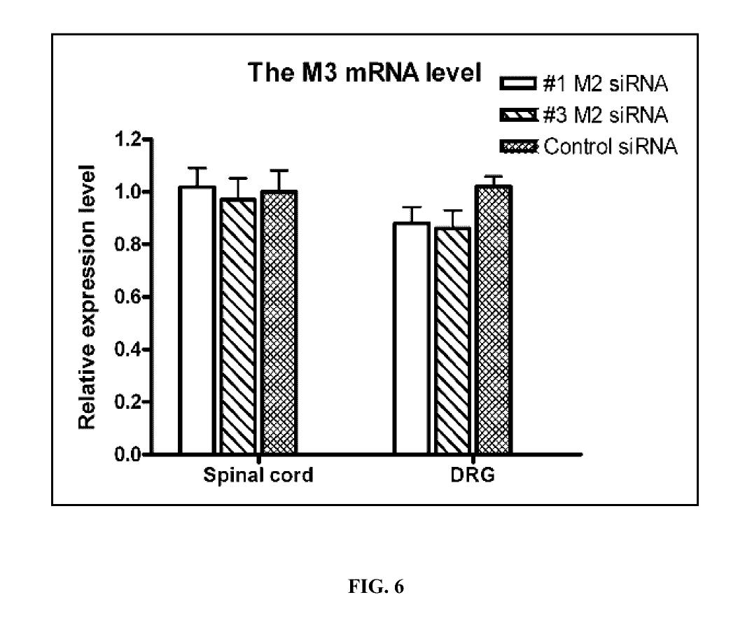

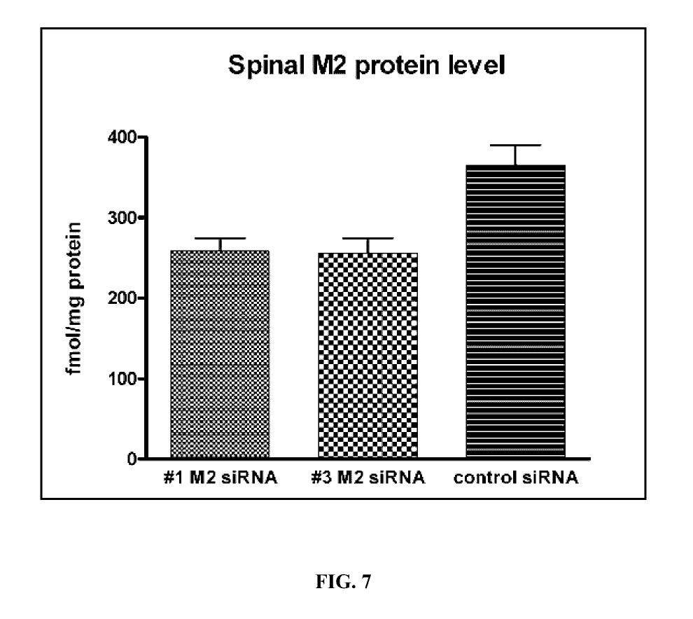

A study was then conducted to examine the effect of intrathecal delivery of siRNA targeted to the M2 mAChR in the spinal cord on the M2 mRNA and protein levels. The mRNA level in the DRG and dorsal spinal cord was measured with quantitative RT-PCR. The mAChR protein in the dorsal spinal cord was quantified using immunoprecipitation followed by the [³H]QNB (a mAChR ligand) binding assay. Intrathecal injection of M2 siRNA-chitosan complex (5 once every other day for 6 days) caused a large reduction (˜60%) of the mRNA level of the M2 mAChR subtype (but not the M3 subtype) in the lumbar DRG and dorsal spinal cord (FIG. 5 and FIG. 6). Furthermore, this treatment strategy effectively reduced the M2 mAChR protein in the dorsal spinal cord (FIG. 7). These findings suggest that siRNA-chitosan is highly effective and specific in the knocking down of the targeted gene and protein in the spinal cord and DRGs.

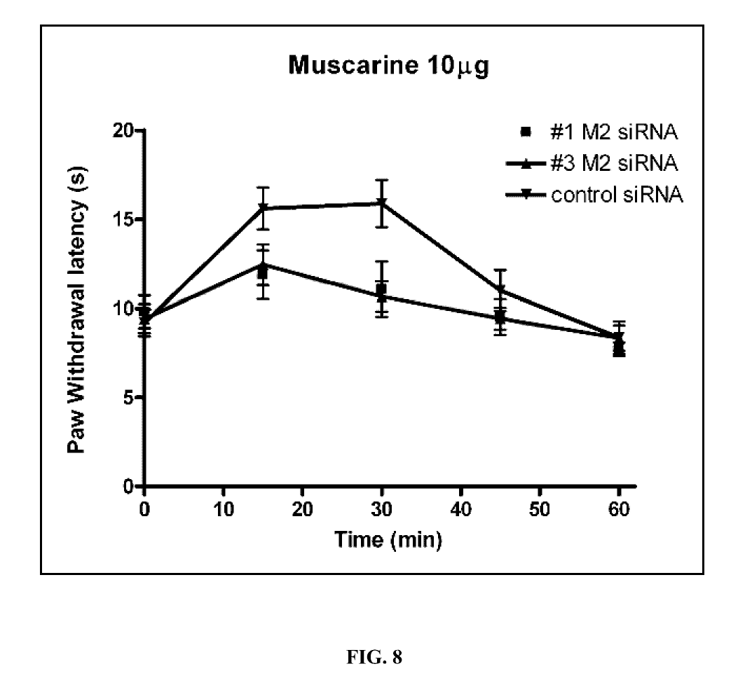

Finally, a study was conducted to examine the extent to which the M2 mAChR subtype in the spinal cord is involved in the analgesic effect produced by the mAChR agonist muscarine. To determine sensitivity to noxious heat, rats were placed in plexiglass enclosures on a transparent glass surface maintained at 30° C. and allowed to acclimate for 30 min. A thermal testing apparatus, consisting of a heat-emitting projector lamp and an electronic timer, was used. The device was activated after the lamp was placed directly beneath the plantar surface of the hindpaw. The paw withdrawal latency in response to the radiant heat was recorded by a digital timer. A cutoff of 30 s was used to prevent potential tissue damage. It was found that the analgesic effect produced by intrathecal injection of muscarine was largely attenuated in siRNA-treated rats (FIG. 8). Thus, the M2 mAChR subtype in the spinal cord plays an important role in the regulation of pain transmission. Intrathecal delivery of siRNA using chitosan particles of the present invention is an effective genetic approach to selectively knock down a desired phenotype in the spinal cord and sensory neurons.

### Example 4

### Anti-Angiogenic Effects of EZH2 Gene Silencing in Ovarian Carcinoma Methods

Cell Lines and Culture.

The HeyA8 and SKOV3ip1 human epithelial ovarian cancer cells were maintained as described previously (Landen et al., 2005). The derivation and characterization of the murine ovarian endothelial cells (MOEC) has been described previously (Langley et al., 2003). The EAhy926 endothelial hybridoma cell line was provided by Dr. Robert Danner, CCMD, NIH, and was maintained as described previously, with sodium hypoxanthine and thymidine (HT) supplement (Invitrogen, Carlsbad, Calif.) instead of sodium hypoxanthine aminopterin and thymidine (HAT) supplement (Invitrogen) (Ptasinska et al., 2007). HUVEC cells were purchased from Cambrex (Walkersville, Md.) and maintained with heparin and gentamicin/amphotericin-B, as previously described (Ptasinska et al., 2007).

EZH2 Promoter Construct.

The EZH2 promoter was amplified by PCR from the Roswell Park Cancer Institute (RPCI) human BAC library 11, Clone-ID RP11-992C19 purchased from the Children's Hospital Oakland Research Institute (Oakland, Calif.), and then cloned into the pGL3-Basic Vector (Promega Corp., Madison, Wis.). The EZH2 promoter construct was amplified using primers (Table 2) with XhoI and HindIII restriction endonuclease sites added to the ends. Purified PCR product was then cloned upstream of the luc+ gene in the pGL3-Basic Vector (Promega Corp.) using XhoI and HindIII.

TABLE 2
Primers and siRNA sequences used

| | |
|---|---|
| EZH2 promoter: | 5′-GATACTCGAGGTCGGGAGTTCGAGACCA-3′(forward) (SEQ ID |
| | NO: 3) |
| | 5′-GTTTAAGCTTACTCGCGTTGTTCCCGCG-3′ (reverse) (SEQ ID |
| | NO: 4) |

Real time qRT-PCR and ChIP assay:

| | |
|---|---|
| Murine EZH2: | 5′-GCTGAGCGTATAAAGACACC-3′ (forward) (SEQ ID NO: 5) |
| | 5′-TCTACATCCTCAGTGGGAAC-3′ (reverse) (SEQ ID NO: 6) |
| Human EZH2: | 5′-TCATGCAACACCCAACAC-3′ (forward) (SEQ ID NO: 7) |
| | 5′-CACAACCGGTGTTTCCTC-3′ (reverse) (SEQ ID NO: 8) |
| Murine VASH1: | 5′-CATCAGGGAGCTGCAGTACA-3′ (forward) (SEQ ID NO: 9) |
| | 5′-CCCAGCTTCACCTTCTTCAG-3′ (reverse) (SEQ ID NO: 10) |
| Human VASH1: | 5-CATGGGAGGGCTTGATGAAGG-3′ (forward) (SEQ ID NO: 11) |
| | 5′-CAAGGTCAGCATGGACTAGGC-3′ (reverse) (SEQ ID NO: 12) |
| SiRNA target sequences: | |
| Nonsilencing control siRNA: | 5′-AATTCTCCGA-ACGTGTCACGT-3′ (SEQ ID NO: 13) |
| Human EZH2 siRNA: | 5′-AATGACATGCCGATCTACATG-3′ (SEQ ID NO: 14) |
| Murine EZH2 siRNA: | 5′-GCACAAGTCATCCCGTTAATT-3′ (SEQ ID NO: 15). |

Luciferase Reporter Assay.

Relative activity of the EZH2 promoter in the EAhy926 cell line was determined by luciferase reporter assay. Cells were transfected in low-serum medium (0.5% serum) with the firefly luciferase plasmid, either empty vector (pGL3-Basic) or the EZH2 promoter construct vector (EZH2prom-pGL3-Basic), in 12-well plates using Effectene® Transfection Reagent from Qiagen (Valencia, Calif.). Cells were then maintained in low-serum medium for 18 hours, washed in warm 1× phosphate-buffered saline (PBS), and treated in triplicate at 37° C. for 6 hours. Treatments included recombinant human (rh) EGF (EGF; 25 ng/mL; Invitrogen) and rhVEGF165 (VEGF; 50 ng/mL; Peprotech, Rocky Hill, N.J.), each in fresh medium plus 0.5% serum, fresh complete medium plus 10% serum, and conditioned media from immortalized ovarian surface epithelium (105E120) and from papillary serous ovarian cancer cell lines (OVCA420 and SKOV3). Medium in control wells (pGL3-Basic transfectants) was not changed on the day of treatment. Following treatment, cells were washed briefly in cold 1×PBS and lysates were collected and processed using the Dual-Luciferase® Reporter Assay System (Promega Corp.). Firefly luciferase readings were averaged and normalized to pGL3-Basic control readings for percent fold changes.

Chromatin Immunoprecipitation (ChIP) Assay.

HUVEC were cultured in low serum medium (0.5% serum) for 18 h and then treated with or without VEGF (50 ng/mL) for 6 hours. After treatment, ChIP assays were performed using EZ ChIP® kit (Milllipore, Temecula, Calif.) as described by the manufacturer. Briefly, cross-linked cells were collected, lysed, sonicated and subsequently subjected to immunoprecipitation with EZH2 (Cell signaling) antibody or mouse IgG (mIgG) control. Immunocomplexes were collected with protein G agarose beads and eluted. Cross-links were reversed by incubating at 65° C. DNA then was extracted and purified for PCR using primers (Table 2) corresponding to the 3800 to 3584 base pairs upstream of the VASH1 transcription start site.

Real Time Quantitative RT-PCR.

Relative expression of EZH2 and VASH1 mRNA in HUVEC and MOEC cells was determined by real-time quantitative RT-PCR. Cells were seeded at 1.0×104 cells per well in 96-well plates in complete medium and incubated at 37° C. for 24 hours, and then in low-serum medium (0.5% serum) for 18 hours, minus EGF and VEGF supplements where appropriate. After washing with warm PBS, cells were treated in triplicate at 37° C. for six hours with EGF (25 ng/mL) and VEGF (50 ng/mL), each in fresh medium (lacking supplemental EGF or VEGF) with no serum, fresh complete medium plus 2% serum, and conditioned media. Relative expression of VASH1 mRNA in MOEC cells was determined by transfecting cells with EZH2 mouse siRNA. Samples were collected after 72 hours of transfection. Real-time quantitative RT-PCR was performed using 50 ng total RNA isolated from treated cells using the RNeasy Mini Kit (Qiagen). Primer sequences are given in Table 2. Relative expression values were obtained using the average of three reference genes and the 2^{-ΔΔCT} method as described previously, and normalized to control for percent fold changes (Donninger et al., 2004).

SiRNA Constructs and Delivery.

Nonsilencing control siRNA and EZH2 Hs siRNA were purchased from Qiagen, and EZH2 Mm siRNA was purchased from Dharmacon (Chicago, Ill.). A nonsilencing siRNA that did not share sequence homology with any known human mRNA based on a BLAST search was used as control for target siRNA, and the same sequence with Alexa-555 tag was used to determine the uptake and distribution in tumor and various organs when given in vivo. In vitro transient transfection was performed as described previously (Landen et al., 2005) and cells were harvested to measure EZH2 protein downregulation by Western blot analysis.

Preparation of siRNA-Incorporated Chitosan (siRNA-Chitosan) Nanoparticles.

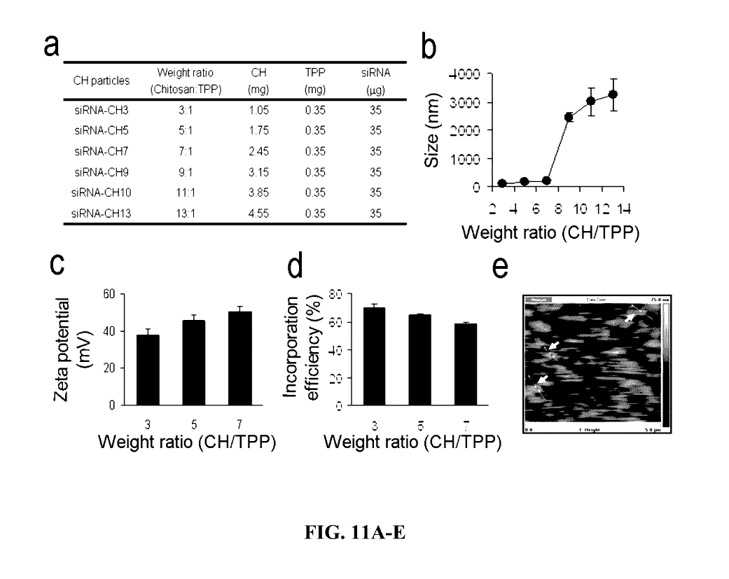

Chitosan (molecular weight 50-190 kDa), sodium tripolyphosphate (TPP), and agarose were purchased from Sigma Co. (St. Louis, Mo.). SiRNA-chitosan nanoparticles were prepared based on ionic gelation of anionic TPP and siRNA with cationic chitosan. The formulation of the siRNA-chitosan nanoparticles is shown in FIG. 11A. Briefly, various concentrations of chitosan solution was obtained by dissolving chitosan in 0.25% acetic acid and nanoparticles were spontaneously generated by the addition of TPP (0.25% w/v) and siRNA (1 μg/μL) to chitosan solution under constant stirring at room temperature. After incubating at 4° C. for 40 min, siRNA-chitosan nanoparticles were collected by centrifugation (Thermo Biofuge, Germany) at 12,000 rpm for 40 minutes at 4° C. The pellet was washed 3 times to remove unbound chemicals or siRNA and siRNA-chitosan nanoparticles were stored at 4° C. until used.

Characteristics of siRNA-Chitosan Nanoparticles.

The size and zeta potential of the siRNA-chitosan nanoparticles were measured by light scattering with a particle size analyzer and Zeta Plus (size and zeta potential analyzer, Brookhaven Instrument Co., CA), respectively. To measure the loading efficiency of siRNA into chitosan nanoparticles, Alexa-555 fluorescent-labeled siRNA was incorporated into chitosan nanoparticles followed by centrifugation at 12,000 rpm for 40 minutes. The fluorescence intensity in the supernatant was measured at 590 nm using fluorescence spectrophotometer (Fluostar Optima, BMG Labtech Inc., Durham, N.C.). Additionally, the morphology of chitosan nanoparticles was confirmed by AFM.

Intracellular Delivery of siRNA.

Intracellular delivery efficiency of the siRNA-chitosan nanoparticles was confirmed using fluorescence microscopy and flow cytometry analysis. HeyA8 cells were incubated in RPMI-1640 serum containing medium at 37° C. After 24 hours, cells were washed and incubated with 2.5 μg of either Alexa-555-labeled siRNA alone or siRNA-chitosan nanoparticles in PBS for 20 minutes at 4° C. Cells were washed carefully with PBS to remove the unbound complexes and either stained with Hoechst 33258 (1.0 μg/mL) for 10 minutes (to stain nuclei blue) and observed under fluorescence microscope (magnification ×200) or subjected to flow cytometry.

Orthotopic In Vivo Model of Ovarian Cancer and Tissue Processing.

Female athymic nude mice (NCr-nu) were purchased from the National Cancer Institute-Frederick Cancer Research and Development Center (Frederick, Md.) and maintained as previously described (Landen et al., 2005). All mouse studies were approved by the M.D. Anderson Cancer Center Institutional Animal Care and Use Committee. Preparation of cells for in vivo injections, determination of uptake of single-dose fluorescent siRNA in tumor tissue and other organs or silencing potential were done as previously described (Landen et al., 2005). At various time points (after 15, 24 hours, 3, 5 and 7 days of siRNA injection), tumors and other organs were harvested. Tissue specimens were fixed either with formalin or OCT (optimum cutting temperature; Miles, Inc., Elkhart, Ind.) or were snap frozen.

To assess tumor growth for long-term therapy experiments, treatment began 1 week after intraperitoneal injection of tumor cells. Mice were divided into 4 groups (n=10 mice per group): (a) control siRNA/CH, (b) EZH2 Hs siRNA/CH (c) EZH2 Mm siRNA/CH, and (d) EZH2 Hs siRNA/CH plus EZH2 Mm siRNA/CH. Each siRNA was given twice weekly at a dose of 150 μg/kg body weight. Treatment continued until mice became moribund (typically 4 to 5 weeks following tumor-cell injection) in any group. At the time of sacrifice, mouse weight, tumor weight, number of nodules, and distribution of tumors were recorded. The individuals who performed the necropsies, tumor collections, and tissue processing were blinded to the treatment group assignments.

Immunofluorescence and Confocal Microscopy.

Determination of uptake of Alexa-555 siRNA/CH by tumor tissues and evaluation of vasculature were performed as described earlier (Landen et al., 2005). Localization of EZH2 and CD31 was performed using frozen tissue. Tumors collected after 48 hours of single injection of control siRNA/CH, or EZH2 Hs siRNA/CH, or EZH2 Mm siRNA/CH, or EZH2 Hs siRNA/CH plus EZH2 Mm siRNA/CH and stained for CD31 and EZH2. Staining for CD31 and desmin was done as described previously (Lu et al., 2007). Pericyte coverage was determined by the percent of vessels with 50% or more coverage by the green fluorescence of associated desmin-positive cells in 5 random fields at ×200 magnification for each tumor.

Western Blot Analysis.

Western blot analysis for EZH2 expression for in vivo samples was performed as previously reported (Landen et al., 2005; Halder et al., 2006). Tumors were collected at various time points (after 24, 48, 72 and 96 hours of single injection of control siRNA/CH, or EZH2 Hs siRNA/CH, or EZH2 Mm siRNA/CH, or EZH2 Hs siRNA/CH plus EZH2 Mm siRNA/CH) and lysed to analyze protein levels using Western blotting.

Immunohistochemical Staining.

Detection of microvessel density was performed using formalin-fixed, paraffin-embedded tumor sections (8 μm thickness) as previously described (Thaker, et al., 2006; Kim et al., 2007) To quantify MVD, the number of blood vessels staining positive for CD31 was recorded in 10 random 0.159 mm² fields at ×200 magnification. All staining was quantified by 2 investigators in a blinded fashion. Immunohistochemistry for EZH2 (1:400 dilution, Zymed, San Francisco, Calif.), CD34 (1:20 dilution, BioGenex Laboratories, San Ramon, Calif.), VEGF (1:100 dilution, Santa Cruz Biotechnology, Inc., Santa Cruz, Calif.) was performed, as described previously (Ali-Fehmi et al., 2005). A combined score that was based on the staining intensity and the percentage of cells stained was used to assign a final score (Ali-Fehmi et al., 2005).

Human Ovarian Cancer Specimens.

Following approval by the Institutional Review Board, 130 paraffin-embedded epithelial ovarian cancer specimens with available clinical outcome data and confirmed diagnosis by a board-certified gynecologic pathologist were obtained from the Karmanos Cancer Institute tumor bank. All patients were diagnosed from 1985 to 2004 following primary cytoreductive surgery. Slides of tumor samples were obtained for EZH2, CD34, and VEGF expression analysis. Clinical variables obtained for correlative analyses included age at diagnosis, tumor stage and grade, and vital status of patients relative to disease-specific survival at the time of chart review.

Statistical Analysis.

Differences in continuous variables such as mean body weight, tumor weight, and proliferation (PCNA) were analyzed using the Mann-Whitney rank sum test. Statistical analyses were performed using SPSS 12.0 for Windows® (SPSS Inc., Chicago, Ill.). A 2-tailed p<0.05 was considered statistically significant. Kaplan-Meier survival plots were generated and comparisons between survival curves were made using the log-rank statistic.

Conditioned Media.

Conditioned media were obtained as follows: IOSE120, OVCA420 and SKOV3 cells were grown in 100 mm culture dishes at 37° C. until 80% confluent. Cells were then washed briefly in warm 1×PBS. Then, 5 mL of low-serum, complete HUVEC cell medium (0.5% serum) was added to the dishes and the cells were incubated at 37° C. for 16 hours. Supernatants (conditioned media) were then collected in a syringe and passed through a 0.45 micron filter and stored at −80° C. until needed.

Gel Retardation Assay.

The incorporation of siRNA into chitosan nanoparticles was determined by 4% agarose gel electrophoresis. Electrophoresis was carried out at a constant voltage of 100 V for 1 hour in 0.5% TAE buffer containing 0.5 μg/mL ethidium bromide (EtBr). The siRNA bands were then visualized under a UV transilluminator (FluorChem 8900, Alpha Innotech, Madison, Wis.).

Stability Assay.

Stability of the siRNA-chitosan nanoparticles in 50% serum was characterized using 4% agarose gel electrophoresis. Either naked siRNA or siRNA-chitosan nanoparticles were mixed in a 1:1 ratio with fresh serum to get the 50% concentration and incubated at 37° C. Aliquots of 20 μL were collected at selected time intervals, loaded onto an agarose gel followed by electrophoresis to visualize intact siRNA.

Pathway Analysis.

Pathway diagrams were generated with the assistance of Pathway Studio software (Ariadne, Rockville, Md.).

Immunofluorescence to Detect Macrophages and Uptake of Alexa-555 siRNA/CH into Organs.

Detection of macrophages and uptake of Alexa-555 siRNA/CH was performed as described previously (Landen et al., 2005). Briefly, tumor and organ tissues were harvested at different time points after single injection of either untagged control siRNA/CH or Alexa-555 siRNA/CH into HeyA8 tumor bearing mice. Tumors were frozen immediately using OCT in liquid nitrogen. Frozen tumor sections (8 μm thickness) were fixed in fresh, cold acetone for 10 minutes, washed 3 times with PBS for 5 minutes and either counterstained with Hoechst to stain nuclei for 10 minutes to check the uptake of siRNA into different organs or blocked with protein block (5% normal horse serum plus 1% normal goat serum in PBS) for 20 minutes at room temperature followed by washing 3 times with PBS. Tissues were incubated with anti-f4/80 primary macrophage antibody (10 μg/mL) at 4° C. overnight followed by secondary goat anti-rat Alexa 488 (4 μg/mL) for 1 hour at room temperature. Tissues were washed 3 times with PBS before and after incubated with secondary antibody and counterstained with Hoechst for 10 minutes. After 3 washes with PBS for 5 minutes, tissues were mounted with mounting medium and examined for macrophages.

Western Blot Analysis.

EZH2 gene silencing by human and mouse targeted EZH2 siRNA in both HeyA8 cells or MOEC was determined by Western blot analysis as previously reported (Landen et al., 2005; Halder et al., 2006). Briefly, cells were transfected with either control or human EZH2, or mouse EZH2 siRNA, collected after 72 hours of transfection and lysate was prepared to perform Western blot analysis to measure EZH2 protein levels.

Immunohistochemical Staining.

Proliferating cell nuclear antigen (PCNA) staining was performed using formalin-fixed, paraffin-embedded tumor sections (8 μm thickness) as previously described (Thaker et al., 2006; Kim et al., 2007). To quantify PCNA expression, the number of positive cells (3, 3′-diaminobenzidine staining) was counted in 10 random 0.159 mm² fields at ×200 magnification. All staining was quantified by two investigators in a blinded fashion.

EZH2 Gene Silencing in MOEC.

Relative expression of EZH2 mRNA in MOEC was determined by transfecting cells with control or EZH2 Mm siRNA and harvested after 72 hours of transfection. Real-time quantitative RT-PCR was performed using 50 ng total RNA isolated from treated cells using the RNeasy Mini Kit (Qiagen). Primer sequences are given in the Supplementary Table 3. Relative expression values were obtained using the average of 3 reference genes and the 2^{-ΔΔCT} method as described previously, and normalized to control for percent fold changes (Donninger et al., 2004).

### Results

EZH2 Expression in Human Ovarian Carcinoma.

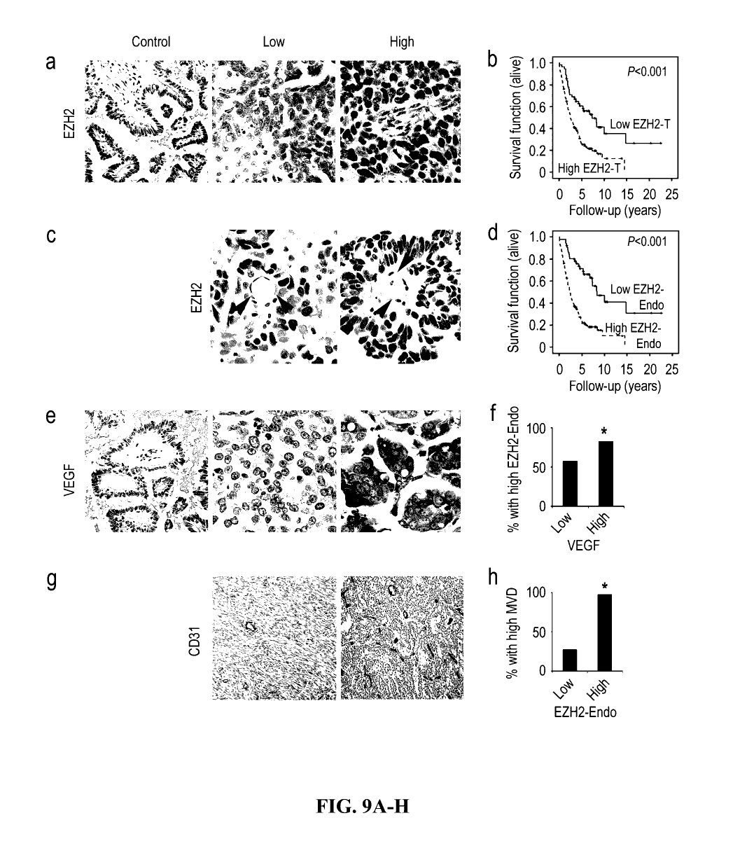

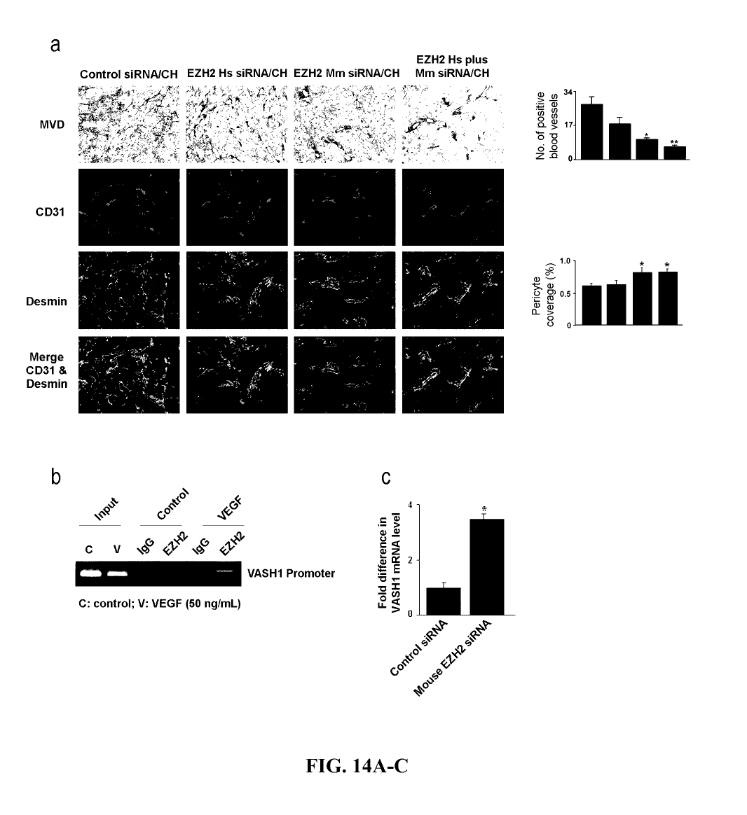

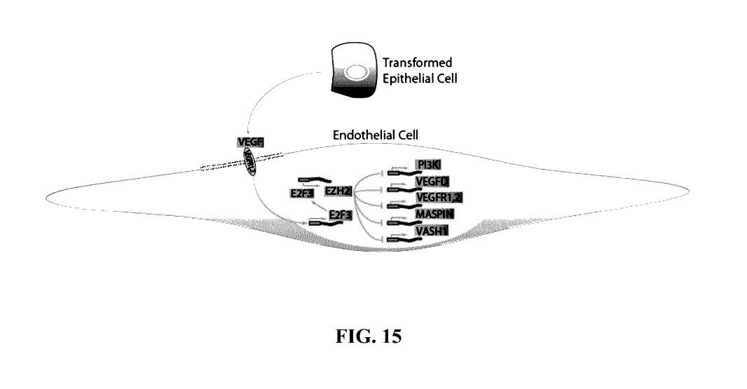

The clinical significance of EZH2 in 130 epithelial ovarian cancers was first examined. Increased tumoral EZH2 (EZH2-T) expression was noted in 66% of samples and increased expression in the vasculature (EZH2-Endo) was noted in 67% of the samples (FIG. 9A). Increased expression of EZH2-T and EZH2-Endo was significantly associated with high stage (p values<0.001) and high grade (p values<0.05; Table 3) disease. Increased EZH2-T was significantly associated with decreased overall survival (median 2.5 years vs. 7.33 years, p values<0.001; FIG. 9B). Similarly, EZH2-Endo was predictive of poor overall survival (2.33 vs. 8.33 years, p<0.001; FIGS. 9C and 1D). On the basis of pathway-analysis predictions from the genomic profiling data comparing endothelial cells from epithelial ovarian cancer with those from normal ovarian tissues5 (FIG. 15), potential associations between EZH2 expression, VEGF expression and microvessel density (MVD) was next examined. Increased VEGF expression was strongly associated with increased EZH2-Endo expression (p<0.001; FIGS. 9E and 9F). Moreover, increased EZH2-Endo expression was significantly associated with high MVD counts in the tumor (p<0.001; FIGS. 9G and 9H).

TABLE 3
Association of clinical and demographic features
with EZH2 in epithelial ovarian carcinoma

| | | | | EZH2-Endo | | |
|---|---|---|---|---|---|---|
| | EZH2-T overexpression | | | overexpression | | |
| | No | Yes | p-value | No | Yes | p-value |
| Mean Age (Yrs) | 59.8 (range 37-89 years) | | | | | |
| Stage | | | | | | |
| Low (I/II) | 20 | 9 | <0.001 | 20 | 9 | <0.001 |
| High (III-IV) | 41 | 108 | <0.001 | 37 | 112 | <0.001 |
| Grade | | | | | | |
| Low | 9 | 7 | 0.048 | 10 | 6 | 0.005 |
| High | 52 | 112 | 0.048 | 37 | 112 | 0.005 |
| Histology | | | | | | |
| Serous | 22 | 18 | 0.002 | 22 | 1 | <0.001 |
| Others | 39 | 100 | 0.002 | 35 | 104 | <0.001 |

VEGF Increases EZH2 Levels in Endothelial Cells.

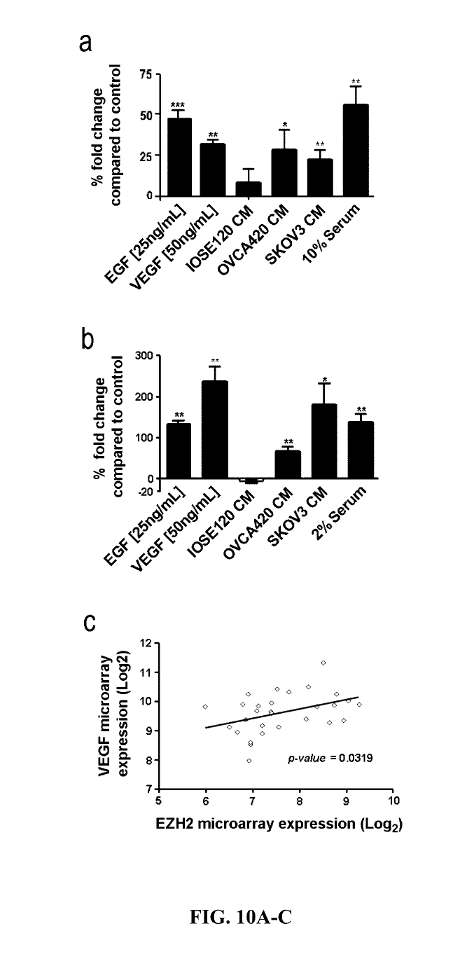

On the basis of the observations from clinical samples, it was next asked whether VEGF could directly regulate EZH2 levels in endothelial cells. For these experiments, EAhy926 hybridoma endothelial cells were cotransfected with the Renilla luciferase plasmid and firefly luciferase plasmid either with or without the EZH2 promoter construct. Cells were then treated with VEGF, EGF, or conditioned media from ovarian cancer cell lines. EZH2 promoter activity was determined by dual-luciferase assay. There was a significant increase in EZH2 promoter activity in endothelial cells in response to VEGF, EGF, and the conditioned media (FIG. 10A). In order to examine changes in EZH2 message, HUVECs were treated as indicated above and expression of EZH2 mRNA was examined using quantitative real time RT-PCR. Control values were normalized using 3 housekeeping genes. EZH2 mRNA expression levels were induced (by 130-240% fold change compared to control) in endothelial cells in response to VEGF, EGF, or the conditioned media (FIG. 10B). To examine the relationship between EZH2 and VEGF in human samples, the expression levels of both genes in 29 microdissected high-grade, serous papillary ovarian cancers was examined. Pearson's analysis showed a significant correlation between EZH2 and VEGF levels (p=0.03; FIG. 10C).

Characteristics of siRNA-Chitosan Nanoparticles.

It was previously demonstrated that EZH2 gene silencing impairs endothelial cell migration and tube formation (Lu et al., 2007). In this example, it was sought to determine whether EZH2 silencing in vivo would affect tumor growth and angiogenesis. Before conducting the EZH2 targeted in vivo experiments, chitosan nanoparticles for systemic delivery of siRNA into both tumor cells and tumor-associated vasculature were developed and characterized. Several formulations of chitosan with siRNA (siRNA-chitosan) were tested (FIG. 11A) and the size of the nanoparticles was examined by light-scattering with a particle-size analyzer (FIG. 11B). At chitosan:TPP ratios varying from 3:1 to 7:1, the sizes of siRNA-chitosan nanoparticles were approximately 100 to 200 nm. However, beyond the 9:1 ratio, the sizes of chitosan nanoparticles were substantially higher (FIG. 11B). Therefore, siRNA/CH3, CH5, and CH7 nanoparticles were selected for further characterization. The zeta potentials of these 3 nanoparticles showed a slight positive charge (FIG. 11C). The incorporation efficiency of siRNA into chitosan nanoparticles was next tested, and the 3:1 ratio (chitosan:TPP) nanoparticles showed the highest (>75%) incorporation efficiency (FIG. 11D). Additionally, the morphology and size of the siRNA/CH3 nanoparticles was determined using atomic force microscopy (AFM), which indicated that the morphology of the nanoparticles was spherical and the size was <150 nm (FIG. 11E). Therefore, for all subsequent experiments, the siRNA/CH3 nanoparticles were used due to their small size, slight positive charge, and high incorporation efficiency of siRNA.

Gel electrophoresis was performed to confirm siRNA incorporation into chitosan nanoparticles. In contrast to “naked” siRNA, the siRNA-chitosan nanoparticles are located on top of the gel due to the positive charge of chitosan, indicating that siRNA is well incorporated into the nanoparticles (FIG. 16A). To examine the stability of the siRNA in chitosan nanoparticles, electrophoretic migration in the presence of 50% serum was examined. As expected, naked siRNA was degraded over 12 to 24 hours in serum containing media; however, the chitosan nanoparticles protected siRNA from such degradation (FIG. 16B).

Intracellular Delivery of siRNA.

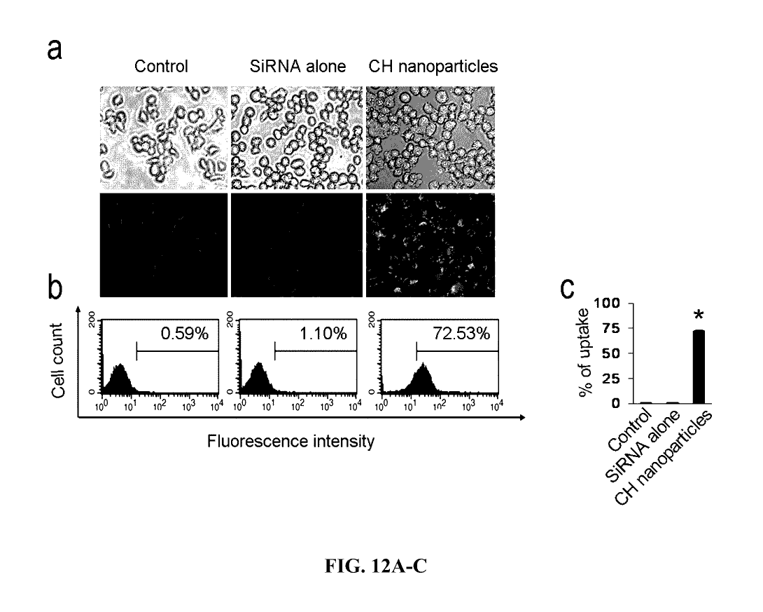

It was tested whether chitosan nanoparticles would permit in vitro delivery of siRNA into cancer cells. Chitosan nanoparticles containing Alexa-555 labeled siRNA showed much higher binding efficiency than “naked” siRNA (FIG. 12A). In addition, the intracellular delivery efficiency of siRNA-chitosan nanoparticles was 72-fold higher than that of “naked” siRNA alone (p<0.001; FIGS. 12B and 12C). Presumably, the protonated amine groups of chitosan nanoparticles allow transport across cellular membranes and subsequent endocytosis into cells (Aigner, 2007).

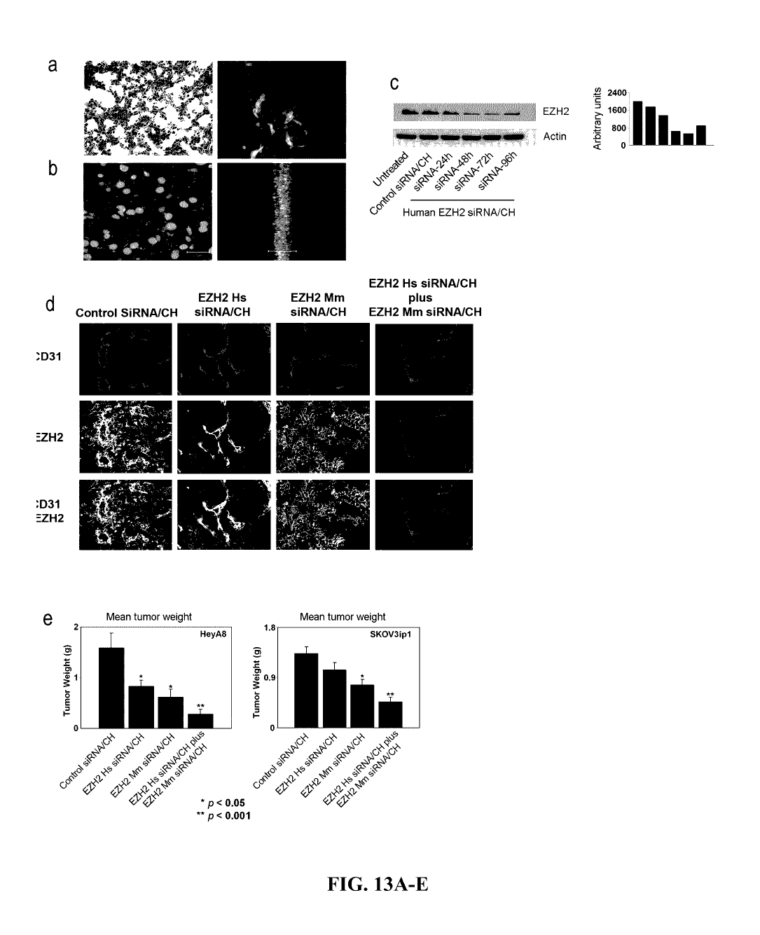

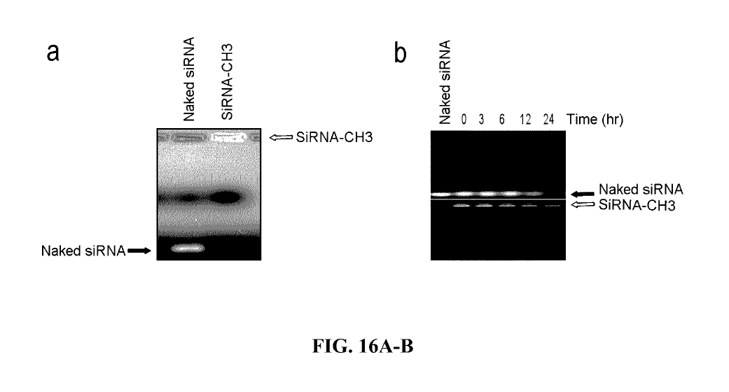

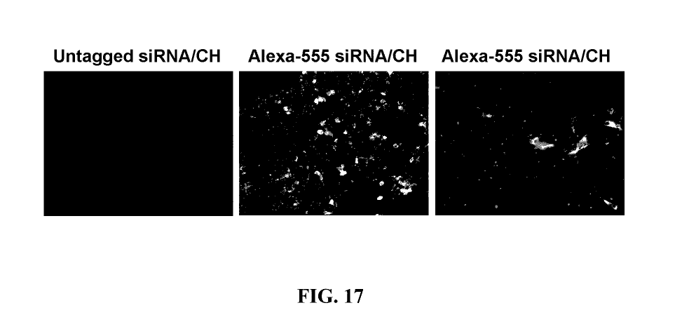

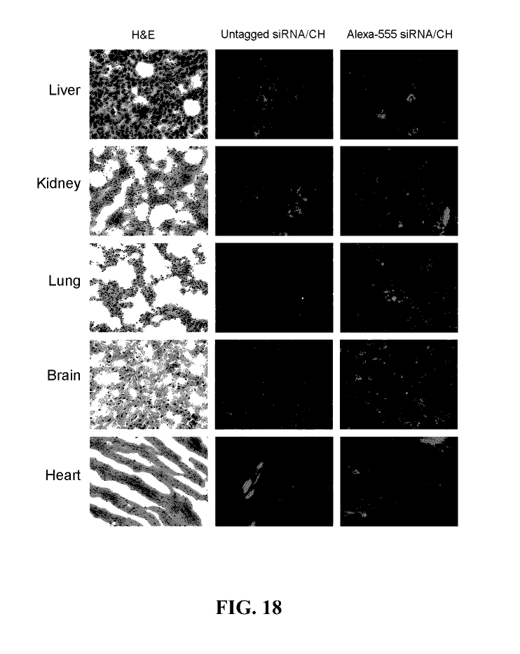

Prior to performing proof-of-concept in vivo efficacy studies, the efficiency of siRNA delivery into orthotopic ovarian tumors was tested. Non-silencing siRNA labeled with Alexa-555 was incorporated into chitosan nanoparticles and injected intravenously (i.v.) into mice bearing HeyA8 orthotopic tumors (17 days after intraperitoneal inoculation of tumor cells). Tumors were harvested at 15 hours and 3, 5 and 7 days (3 mice per time point) following injection and examined for extent of siRNA delivery. At all time points, punctated emissions of the siRNA were noted in the perinuclear regions of individual cells. SiRNA was noted in >80% of fields examined following a single intravenous injection. To confirm delivery of siRNA in the vasculature, slides were also stained for CD31. Indeed, siRNA was delivered into the tumor-associated endothelial cells, suggesting potential applications for targeting the tumor vasculature (FIG. 13A). To confirm intracellular delivery of siRNA, 3-dimensional reconstructions of the tumors were created using confocal microscopy. Lateral views of the optical sections clearly demonstrated the presence of siRNA within the tumor cells (FIG. 13B). However, very little siRNA was taken up by macrophages as determined by labeling tissues with f4/80 (FIG. 17). To examine the delivery of siRNA into other organs, sections of liver, lung, kidney, heart, spleen and brain were also examined, and siRNA delivery was detected in most of these organs (FIG. 18).

EZH2 Gene Silencing with EZH2 siRNA-Chitosan Nanoparticles.

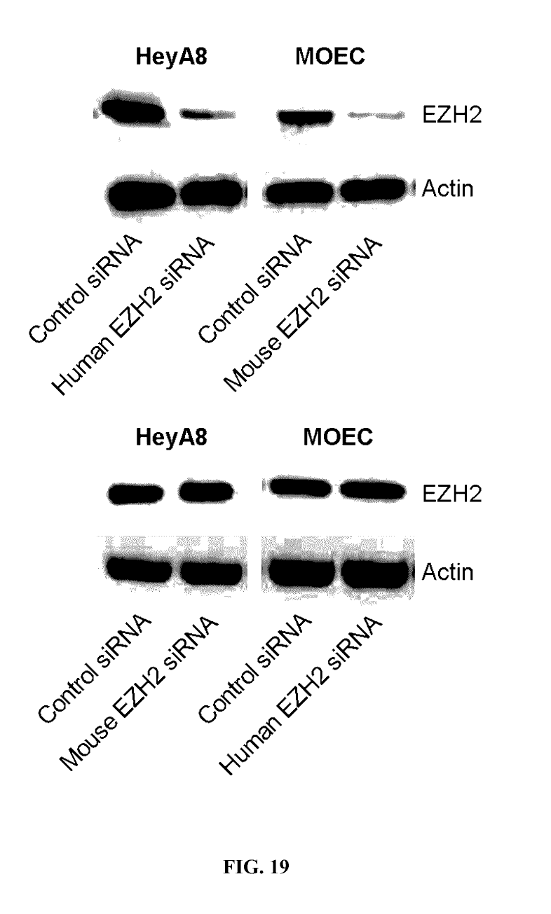

To examine the in vivo effects of EZH2 gene silencing on tumor growth, EZH2 siRNA directed to either the human (tumor cells; EZH2 Hs siRNA/CH) or mouse (endothelial cells; EZH2 Mm siRNA/CH) sequence was utilized. The specificity of siRNA was confirmed by testing each siRNA in both mouse endothelial (MOEC) and human tumor (HeyA8) cells (FIG. 19). Following i.v. injection of either control siRNA/CH, EZH2 Hs siRNA/CH, EZH2 Mm siRNA/CH, or the combination of EZH2 targeted siRNAs into HeyA8 tumor-bearing mice (n=3 mice per group at each time point), tumors were harvested at different time points and examined for EZH2 protein levels. EZH2 levels were decreased by 24 hours following single injection of EZH2 Hs siRNA/CH with return of expression to baseline expression levels after 96 hours (FIG. 13C). To determine the localization of EZH2 silencing following siRNA/CH administration, dual immunofluorescence staining for EZH2 and CD31 was performed. This experiment further demonstrated that EZH2 Hs siRNA/CH resulted in EZH2 silencing in the tumor cells whereas EZH2 Mm siRNA/CH silenced EZH2 only in the tumor endothelial cells (FIG. 13D).

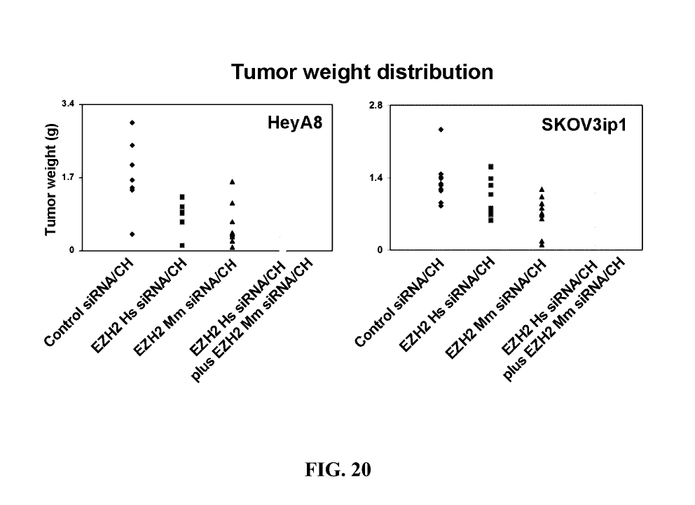

To determine the therapeutic efficacy of EZH2 gene silencing, a well-characterized orthotopic model of ovarian carcinoma was utilized. Seven days following injection tumor cells into the peritoneal cavity, mice were randomly allocated to 1 of 4 groups of 10 mice each: 1) control siRNA/CH, 2) EZH2 Hs siRNA/CH, 3) EZH2 Mm siRNA/CH and 4) combination of EZH2 Hs siRNA/CH plus EZH2 Mm siRNA/CH. Mice were sacrificed when animals appeared moribund due to significant tumor burden (4 to 5 weeks after cell injection depending on the cell line). As shown in FIG. 13E and FIG. 20, in both models, treatment with EZH2 Mm siRNA/CH resulted in a significant decrease in tumor burden compared to control siRNA/CH (62% reduction in HeyA8; p<0.02 and 40% reduction in SKOV3ip1, p<0.03). EZH2 Hs siRNA/CH as a single-agent had modest effects on tumor growth (p<0.04 for HeyA8; and p<0.05 for SKOV3ip1) compared with control siRNA/CH. However, the greatest reduction was observed with the combination of EZH2 Hs siRNA/CH plus EZH2 Mm siRNA/CH (83% reduction in HeyA8, p<0.001 and 65% reduction in SKOV3ip1, p<0.001). To test for potential off-target effects, the efficacy of 3 additional mouse EZH2 siRNA sequences with similar effects on tumor growth was tested (data not shown).

To evaluate the effects of EZH2 on other parameters of tumor growth, tumor incidence and number of nodules were examined (Table 4). The combination of EZH2 Hs siRNA/CH plus EZH2 Mm siRNA/CH resulted in a significant reduction in tumor nodules in both HeyA8 (p=0.002 vs. control siRNA treated group) and SKOV3ip1 tumors (p=0.004 vs. control siRNA treated group). The decrease in tumor burden occurred despite having comparable tumor incidence. The mean mouse body weight was similar among the different groups, suggesting that feeding and drinking habits were not affected.

TABLE 4
Characteristics of tumors after treated with
human and mouse EZH2 siRNA/CH

| | | Median no. | |
|---|---|---|---|
| | | nodules | p-value |
| Cell line | Treatment | (range) | (vs. control) |
| HeyA8 | Control siRNA/CH | 6.5 (3-11) | |
| | EZH2 Hs siRNA/CH | 3.5 (1-11) | 0.05 |
| | EZH2 Mm siRNA/CH | 3.0 (1-9) | 0.05 |
| | EZH2 Hs siRNA/CH plus | 1.5 (1-7) | 0.002 |
| | EZH2 Mm siRNA/CH | | |
| SKOV3iP1 | Control siRNA/CH | 16.0 (11-26) | |
| | EZH2 Hs siRNA/CH | 16.0 (8-27) | ns |
| | EZH2 Mm siRNA/CH | 12.0 (1-11) | 0.05 |
| | EZH2 Hs siRNA/CH plus | 7.5 (2-27) | 0.004 |
| | EZH2 Mm siRNA/CH | | |

Effect of EZH2 Targeting on Tumor Vasculature and Proliferation.

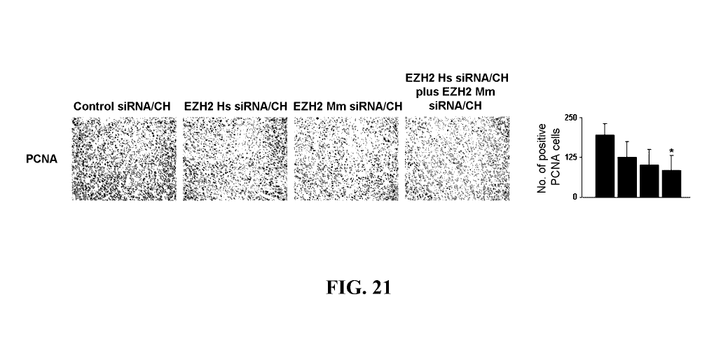

To determine the potential mechanisms underlying the efficacy of EZH2 silencing on ovarian tumors, its effects on several biological end points were examined, including MVD, pericyte coverage (desmin) and cell proliferation (PCNA). EZH2 Mm siRNA/CH and the combination therapy groups had significantly lower microvessel density (FIG. 14A) compared to the EZH2 Hs siRNA/CH and control siRNA/CH treated tumors. Pericyte coverage was increased in EZH2 Mm siRNA/CH and the combination groups compared to other 2 groups, suggesting greater vascular maturation (FIG. 14A). Combination treatment with EZH2 Hs siRNA/CH and EZH2 Mm siRNA/CH also resulted in a significant reduction in cell proliferation (FIG. 21).

EZH2 Silencing Increases VASH1 mRNA Expression in Endothelial Cells.

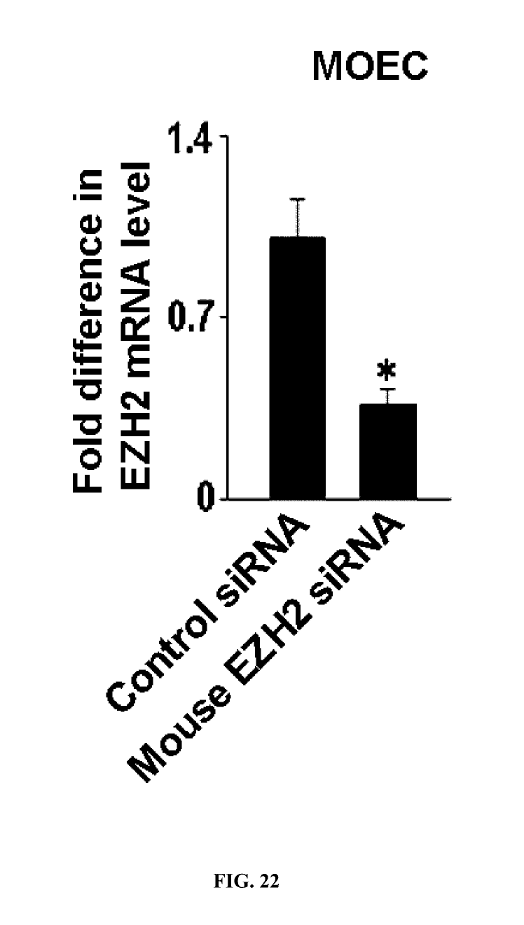

To determine the mechanism by which EZH2 silencing could induce anti-angiogenic effects, a whole genome ChIP-on-ChIP analysis was performed. The findings indicate that an anti-angiogenic gene, vasohibin (VASH1) directly binds to EZH2. To validate this finding, ChIP assay of EZH2 for the VASH1 promoter in endothelial cells was performed in the presence or absence of VEGF (FIG. 14B), which confirmed direct EZH2 binding to the VASH1 promoter. Next, the EZH2 gene in mouse ovarian endothelial cells (MOEC) was silenced using siRNA (FIG. 22), which resulted in a 2.8 fold increase in VASH1 (FIG. 14C). The experiments provide direct explanation for the anti-angiogenesis effects observed in response to EZH2 gene silencing.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,282,351
U.S. Pat. No. 4,659,774
U.S. Pat. No. 4,682,195
U.S. Pat. No. 4,683,202
U.S. Pat. No. 4,806,474
U.S. Pat. No. 4,816,571
U.S. Pat. No. 4,870,287
U.S. Pat. No. 4,959,463
U.S. Pat. No. 5,141,813
U.S. Pat. No. 5,214,136
U.S. Pat. No. 5,223,618
U.S. Pat. No. 5,264,566
U.S. Pat. No. 5,378,825
U.S. Pat. No. 5,428,148
U.S. Pat. No. 5,446,137
U.S. Pat. No. 5,466,786
U.S. Pat. No. 5,470,967
U.S. Pat. No. 5,539,082
U.S. Pat. No. 5,554,744
U.S. Pat. No. 5,574,146
U.S. Pat. No. 5,602,240
U.S. Pat. No. 5,602,244
U.S. Pat. No. 5,610,289
U.S. Pat. No. 5,614,617
U.S. Pat. No. 5,623,070
U.S. Pat. No. 5,645,897
U.S. Pat. No. 5,652,099
U.S. Pat. No. 5,670,663
U.S. Pat. No. 5,672,697
U.S. Pat. No. 5,681,947
U.S. Pat. No. 5,700,922
U.S. Pat. No. 5,705,629
U.S. Pat. No. 5,708,154
U.S. Pat. No. 5,714,331
U.S. Pat. No. 5,714,606
U.S. Pat. No. 5,719,262
U.S. Pat. No. 5,736,336
U.S. Pat. No. 5,739,169
U.S. Pat. No. 5,760,395
U.S. Pat. No. 5,763,167
U.S. Pat. No. 5,766,855
U.S. Pat. No. 5,773,571
U.S. Pat. No. 5,777,092
U.S. Pat. No. 5,786,461
U.S. Pat. No. 5,792,847
U.S. Pat. No. 5,801,005
U.S. Pat. No. 5,824,311
U.S. Pat. No. 5,830,880
U.S. Pat. No. 5,846,945
U.S. Pat. No. 5,858,988
U.S. Pat. No. 5,859,221
U.S. Pat. No. 5,872,232
U.S. Pat. No. 5,886,165
U.S. Pat. No. 5,891,625
U.S. Pat. No. 5,908,845
U.S. Pat. No. 6,506,559
U.S. Pat. No. 6,573,099
U.S. Pat. No. 6,673,611
U.S. Appln. Ser. 117,363
U.S. Patent Pubn. 2002/0168707
U.S. Patent Pubn. 20020170849
U.S. Patent Pubn. 2003/0051263
U.S. Patent Pubn. 2003/0055020
U.S. Patent Pubn. 2003/0159161
U.S. Patent Pubn. 20030129730
U.S. Patent Pubn. 2004/0019001
U.S. Patent Pubn. 2004/0064842
U.S. Patent Pubn. 2004/0265839
U.S. Patent Pubn. 20040247632
U.S. Patent Pubn. 20050042735
U.S. Patent Pubn. 20050226938
U.S. Patent Pubn. 20050245482
U.S. Patent Pubn. 20060094666
U.S. Patent Pubn. 20060189573
U.S. Patent Pubn. 20060277632
U.S. Patent Pubn. 20070116767
U.S. Patent Pubn. 20070167400
U.S. Patent Pubn. 20070311468
Aigner, Curr. Opin. Molec. Therap., 9:345-352, 2007.
Ali-Fehmi et al., Amer. J. Obstet. Gynecol., 192:819-825, 2005.
Allan and Peyron, Carbohydr Res., 277(2):257-272, 1995.
Austin-Ward and Villaseca, Revista Medica de Chile, 126(7):838-845, 1998.
Bayomi et al., Pharm. Acta Helv., 73(4):187-192, 1998.
Bukowski et al., Clinical Cancer Res., 4(10):2337-2347, 1998.
Burger et al., J. Clin. Oncol., 25:5165-5171, 2007.
Cavalli and Paro, Curr. Opin. Cell Biol., 10:354-360, 1998.
Cha et al., Science (NY), 310:306-310, 2005.
Christodoulides et al., Microbiology, 144(Pt 11):3027-3037, 1998.
Davidson et al., J Immunother 21(5):389-398, 1998.
Domard and Cartier, Int. J Biol. Macromol., 11(5):297-302, 1989.
Donninger et al., Oncogene, 23:8065-8077, 2004.
Egholm et al., Nature, 365(6446):566-568, 1993.
European Appln. 266,032
Fire et al., Nature, 391(6669):806-811, 1998.
Folkman, J. Natl. Cancer Instit., 82:4-6, 1990.
Froehler et al., Nucleic Acids Res., 14:5399-5407, 1986.
Genta et al., Drug Dev. Ind. Pharm., 24(8):779-784, 1998.
Gupta and Ravi Kumar, J Mater Sci. Mater Med., 12(9):753-759, 2001.
Halder et al., Clin. Cancer Res., 12:4916-4924, 2006.
Hanibuchi et al., Int. J Cancer, 78(4):480-485, 1998.
Hellstrand et al., Acta Oncologica, 37(4):347-353, 1998.
Hui and Hashimoto, Infection Immun., 66(11):5329-5336, 1998.
Jain et al., Nature Clin. Pract., 3:24-40, 2006.
Ju et al., Gene Ther., 7(19):1672-1679, 2000.
Katas and Alpar, J Control Release, 115(2):216-225, 2006.
Kim et al., Cancer Res., 67:9337-9345, 2007.
Kingston et al., Genes Develop., 10:905-920, 1996.
Ko et al., J Microencapsul., 20(6):791-797, 2003.
Kornberg and Baker, DNA Replication, 2nd Ed., Freeman, San Francisco, 1992.
Kumar, Reactive Funct., Polymers, 46:1-27, 2000.
Landen Jr., et al., Cancer Res., 65:6910-6918, 2005.
Langley et al., Cancer Res., 63:2971-2976, 2003.
Li et al., Virology, 212(1):134-150, 1995.
Liu et al., J Biomed Mater Res A., 2007 (ahead of Eurp Publn)
Lu et al., Cancer Res., 67:1757-1768, 2007.
Mi et al., Advanced Drug Deliv. Rev., 52(1):145-150, 1999.
Mitchell et al., Ann. NY Acad. Sci., 690:153-166, 1993.
Mitchell et al., J Clin. Oncol., 8(5):856-869, 1990.
Morton et al., Arch. Surg., 127:392-399, 1992.
PCT Appln. PCT/EP/01219
PCT Appln. WO 92/20702
Pietras et al., Oncogene, 17(17):2235-2249, 1998.
Ptasinska et al., Faseb J., 21:950-961, 2007.
Qin et al., Proc. Natl. Acad. Sci. USA, 95(24):14411-14416, 1998.
Raman et al., Clin. Cancer Res., 11:8570-8576, 2005.
Ravindranath and Morton, Intern. Rev. Immunol., 7: 303-329, 1991.
Remington's Pharmaceutical Sciences, 18th Ed. Mack Printing Company, pp. 1289-1329, 1990.
Rosenberg et al., Ann. Surg. 210(4):474-548, 1989.
Rosenberg et al., N. Engl. J. Med., 319:1676, 1988.
Sambrook and Russell, Molecular Cloning: A Laboratory Manual 3^{rd} Ed., Cold Spring Harbor Laboratory Press, 2001.
Scheit, In: Synthesis and Biological Function, Wiley-Interscience, New York, 171-172, 1980.
Simon, Curr. Opin. Cell Biol., 7:376-385, 1995.
Song et al., Nature Med. 9:347-351, 2003.
Spannuth et al., Nature Clin. Pract., 5:194-204, 2008.
Thaker et al., Nature Medicine, 12:939-944, 2006.
Varum et al., Carbohydr. Res., 211(1):17-23, 1991.

1.-38. (canceled)

39. A method of treating a subject with ovarian cancer, comprising administering to a subject with ovarian cancer a pharmaceutically effective amount of a composition comprising:
(a) a chitosan; and
(b) a nucleic acid component comprising a nucleic acid that inhibits the expression of a gene that encodes EZH2.

40. The method of claim 39, wherein the nucleic acid component comprises a siRNA or a nucleic acid encoding a siRNA, wherein the siRNA inhibits the expression of a gene that encodes EZH2 in the subject.

41. The method of claim 39, wherein the composition further comprises a lipid.

42. The method of claim 41, wherein the lipid is cholesterol, phosphatidylcholine, or phosphatidylethanolamine.

43. The method of claim 39, wherein the composition further comprises a polyphosphate anion of formula (I): 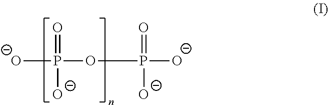
wherein n is an integer ranging from 2-10.

44. The method of claim 39, wherein the subject is a human subject.

45. The method of claim 39, further comprising administering an additional anticancer therapy to the subject.

46. The method of claim 45, wherein the additional anticancer therapy is chemotherapy, radiation therapy, surgical therapy, immunotherapy, gene therapy, or a combination thereof.

47. The method of claim 45, wherein the additional anticancer therapy is a VEGF inhibitor.

48. The method of claim 39, wherein the composition is administered to the patient intravenously, intraperitoneally, intratracheally, intratumorally, intramuscularly, endoscopically, intralesionally, percutaneously, subcutaneously, regionally, or by direct injection or perfusion.

49. A method to inhibit angiogenesis of an ovarian cancer, comprising contacting said cancer with a composition comprising:
(a) a chitosan; and
(b) a nucleic acid component comprising a nucleic acid that inhibits the expression of a gene that encodes EZH2,
wherein angiogenesis of the ovarian cancer is inhibited.

50. The method of claim 49, wherein the ovarian cancer is in a human subject.

51. (canceled)

52. The method of claim 49, wherein the composition further comprises cholesterol.

53. The method of claim 49, wherein the composition further comprises tripolyphosphate.

54.-60. (canceled)

61. The method of claim 49, wherein the nucleic acid component comprises a siRNA or a nucleic acid encoding a siRNA, wherein the siRNA inhibits the expression of a gene that encodes EZH2 in the subject.
